(19) United States
(12) Patent Application Publication
(10) Pub. No.: US 2013/0203145 A1
(43) Pub. Date: Aug. 8, 2013

(54) Continuous Flow Bioreactor for Magnetically Stabilized Three-Dimensional Tissue Culture

(71) Applicants: Christopher R. Lambert, Hudson, MA (US); W. Grant McGimpsey, Hudson, MA (US); Raymond L. Page, Southbridge, MA (US); Tanja Dominko, Southbridge, MA (US)

(72) Inventors: Christopher R. Lambert, Hudson, MA (US); W. Grant McGimpsey, Hudson, MA (US); Raymond L. Page, Southbridge, MA (US); Tanja Dominko, Southbridge, MA (US)

(21) Appl. No.: 13/583,146

(22) PCT Filed: Mar. 8, 2011
(86) PCT No.: PCT/US11/27579

§ 371 Date: Dec. 10, 2012

Related U.S. Application Data
(60) Provisional application No. 61/311,731, filed on Mar. 8, 2010.

(51) Int. Cl. C12N 13/00

(52) U.S. Cl. CPC C12N 13/00
USPC 435/173.1; 435/289.1

(57) ABSTRACT

The invention provides methods for rapid, continuous generation of cells and cell products using magnetically stabilized three-dimensional tissue culture. The invention also pertains to a continuous flow self-regulating closed system bioreactor system for magnetically stabilized three-dimensional tissue culture. The methods described here do not use traditional solid scaffolding for cell culture.

### RELATED APPLICATION

This application claims the benefit of U.S. Provisional Application No. 61/311,731, filed on Mar. 8, 2010. The entire teachings of the above application are incorporated herein by reference.

### BACKGROUND OF THE INVENTION

Cell culture is typically performed on flat two-dimensional surfaces for practical reasons. Although the coating of such surfaces can mimic the biochemical environment of the extracellular matrix of live tissues, the geometry of such a system confines both the ability to directly compare the behavior of these cells with those in living tissue and the ability to generate tissue. Cells growing on a two-dimensional surface cannot interact with other cells in all directions as they would in any tissue of a living organism.

Tissue engineering has become increasingly important in the replacement of damaged tissues and organs of patients resulting from injury or disease. However growing cells on a three-dimensional porous substrate or scaffold has its own limitations. Building of the scaffold either from processed natural products or biomaterials is challenging especially in those cases where more than one cell type is required for tissue generation. When more than one type of cell or tissue is needed, directing and controlling of cell binding and growth in the correct formation on the same scaffold is required. Once such a solid scaffold is exposed to the cells, tissue growth or generation is practically left on its own with no outside control, most probably leading to unexpected and unwanted results. Thus, a need exist for tissue engineering without the need for scaffolding that allows for scalable, efficient, costs reduced production of cells, tissues and their products. A dynamic system is needed that mimics in vivo conditions with an apparatus for production of same.

### SUMMARY OF THE INVENTION

Described herein are methods for rapid, continuous generation of cells and differentiated cells, production of cell products (e.g., cellular metabolites) and a method for making three dimensional tissues. Also described is a bioreactor system that delivers culture conditions for sustaining culturing conditions for various methodologies including but not limited to tissue reconstruction, production of biofuel, production of cell and pharmaceutical cell products and cell amplification.

A continuous flow self-regulating closed system bioreactor system (MSCFB) for magnetically stabilized three-dimensional tissue culture is described. The methods described do not use traditional solid scaffolding for cell culture. The methods and apparatus described herein allow for three-dimensional cell and tissue culturing without the use of traditional scaffolding.

In certain aspects, flexible or movable templates are used such as magnetic beads with or without additional scaffolding that could be removed, added, degradable or replaced. The desired movement is controlled externally. This system allows for controlling the binding and growth of different cells and tissue layers in the anatomically desired formation. Such a template is formed by the use of magnetic beads whose movement can be controlled from an external magnetic field. In certain embodiments, the magnetic field in generated from one or more (e.g., 2, 3, 4, 5, 6, 7, 8, or more) magnets (e.g., annular neodymium magnets) and magnetic beads. One is then able to set and change the shape of the template without the need to directly interact with the template and thus risking infection. The magnetic field applies a force thus acting on the cells to manipulate a controlled development and proliferation. For example, it is possible to grow the cells and produce a lumen by controlling media flow around the cell mass under the control of the magnetic field. The normal interactions of the cells to each other are not adversely affected by the magnetic field.

Magnetic beads have been used successfully for several applications, such as enzyme immobilization and cell separation. In certain applications, the magnetic beads are coated with a thin film or alternatively encapsulated in a polymer matrix. For example, the magnetic beads are prepared from iron oxide nanoparticles embedded in micrometer-size (10-100 micron) agarose gel beads. Since agarose gel is a non-adherent substrate, a problem of bead aggregation is avoided. The magnetic beads can be any shape (geometrically shaped or irregular) and rough or smooth depending on the need.

In certain embodiments, the surface of the beads is coated with other materials for a predetermined desired activity such as coating with antibodies for specific cell-attachment or coating with collagen for non-specific attachment. Other coatings are also contemplated such as polymers. The modifications can be for biochemical means, for example, for targeting or cell specific attachment and the like.

By trapping cells on the surface of the beads, the beads and attached cells are arranged in various formations using an external magnetic field, e.g., a system of magnets. A homogeneous magnetic field generated on the beads is dispersed evenly in the medium creating a three-dimensional environment that allows the cells to generate their own extracellular matrix without the need of a solid scaffold. A continuous flow of the growth medium will provide a fresh supply of nutrients and oxygen and provide the mechanical stress factor required, while at the same time removing waste products that will be adequate for the internal tissue layers.

In a first embodiment, a bioreactor for culturing cells is described comprising: a vessel, an apparatus for conveying fluids, magnetic beads having cells attached thereon, and an apparatus for conveying a magnetic field. In a second embodiment, the bioreactor further includes one or more of the following, an apparatus for dialysis, an apparatus for harvesting and an apparatus for photoelectrochemical processing. In a third embodiment of the invention, a system for cultivating cells is described including a bioreactor of the first or second embodiment, magnetic beads having cells attached thereon, an apparatus for conveying a magnetic field, and an apparatus for conveying a continuous flow of fluids, where the cells are controlled by the magnetic field and the flow of fluids. In an aspect of any one of the embodiments of the invention, more than one cell type is cultured. In another aspect, of the first, second or third embodiment, the cells are grown to a predetermined shape.

In third aspect of the embodiments or methods described above, the cells are mammalian cells, for example, epidermis cells, smooth muscle cells, cardiac tissue cells, cells of particular organs, fibroblast cells, stem cells, and the like. In a fourth aspect of the embodiments described above, particular mammalian tissue is cultured.

In a fifth aspect of any one of the embodiments of the invention, the cells are bacteria cells or algae cells for example, cells of Rhodobacter sphaeroides, Synechococcus elongates, Rdodopseudomanas rutiia, Clostridiumm ljungdahlii, and Chlorogleopsis. In certain aspects of these embodiments, more than one cell type is cultured.

In a sixth aspect of the previously described embodiments or aspects, of the methods, systems and bioreactors described herein, the magnetic bead size is between about 100 nm to about 10 μm.

The methods described here in are advantageous in maximizing cell per unit volume to reduce the use of medium while eliminating waste products. The bioreactor, system and methods allow for controlled cost efficient, volume controlled expansion of cells in three dimensional culture systems for easy expansion of cells and tissue.

In a fourth embodiment, a method for culturing cells in a bioreactor is described. The method includes providing cells attached to magnetic beads, acting upon the cells with a fluid, acting upon the cells with a magnetic field and allowing the cells to grow and proliferate. In a first aspect of the fourth embodiment, the cells form a tissue. In a second aspect of the fourth embodiment or the first aspect of the fourth embodiment, the tissue has a lumen. In a third aspect of the fourth embodiment, in the second aspect of the fourth embodiment or the first aspect of the fourth embodiment, the cells are grown without scaffolding. In a fourth aspect of the fourth embodiment, in a third aspect of the fourth embodiment, in the second aspect of the fourth embodiment or the first aspect of the fourth embodiment, the cells are mammalian cells (e.g., lymphocytes, smooth muscle cells, stem cell, cardiac cells and fibroblasts and combinations thereof). In a fifth aspect of the fourth embodiment, in a fourth aspect of the fourth embodiment, in a third aspect of the fourth embodiment, in the second aspect of the fourth embodiment or the first aspect of the fourth embodiment the magnetic beads are coated with a polymer (e.g., agarose). In a six aspect of the fourth embodiment, in a fifth aspect of the fourth embodiment In a fourth aspect of the fourth embodiment, in a third aspect of the fourth embodiment, in the second aspect of the fourth embodiment or the first aspect of the fourth embodiment, the magnetic bead is further modified (e.g., by attachment of a protein (e.g. collagen). In a seventh aspect of the fourth embodiment, in a six aspect of the fourth embodiment, in a fifth aspect of the fourth embodiment In a fourth aspect of the fourth embodiment, in a third aspect of the fourth embodiment, in the second aspect of the fourth embodiment or the first aspect of the fourth embodiment, the cells grow to form tissue rings. In an eighth aspect, in a seventh aspect of the fourth embodiment, in a six aspect of the fourth embodiment, in a fifth aspect of the fourth embodiment In a fourth aspect of the fourth embodiment, in a third aspect of the fourth embodiment, in the second aspect of the fourth embodiment or the first aspect of the fourth embodiment, the magnetic field includes angular magnetic rings.

In a fifth embodiment, a system of producing a cell product, comprising the bioreactor of the invention wherein the cells produce a cellular metabolite is described. In a first aspect of the fifth embodiment, the cellular metabolite is a protein (e.g., cytokine, IL-2).

Also described in a sixth embodiment is a method for culturing cells in a bioreactor, comprising providing cells attached to magnetic beads, acting upon the cells with a fluid, acting upon the cells with a magnetic field, and allowing the cells to grow and proliferate. In a first aspect of the sixth, the method includes one or more cell types being cultured. In a second aspect of the sixth embodiment or the in addition to the first aspect of the sixth embodiment, the culturing of the cells includes a predetermined shape. For example, the cells can be cultured to product a luman or other three dimensional shape. In a third aspect of the sixth embodiment, including the first or second aspect, biochemical modifications can be utilized for appropriate attachment and control of the cells with the magnetic beads.

In a seventh embodiment, a method for making a three dimensional cell tissue includes culturing cells in a bioreactor, providing cells attached to magnetic beads, acting upon the cells with a fluid, acting upon the cells attached to magnetic beads with a magnetic field, and allowing the cells to grow and proliferate into a three dimensional cell tissue.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A is a photograph showing a representative continuous flow-through magnetic bioreactor system assembled in the incubator (left) and CAD schematic drawing of the continuous flow-through magnetic bioreactor (MSCFB) (right). The peristaltic pump generating the flow in the silicone tubing sits outside of the incubator. The source of the growth of the medium is a tissue culture flask that it is sitting flat in order to allow for gas exchange of the growth medium.

FIG. 1B is a photograph showing the magnetically stabilized continuous flow bioreactor (MSCFB) in an alternate configuration.

FIG. 2A is an image of Jurkat cells attached to the larger magnetic agarose beads coated with CD3 antibody after the overnight incubation. The image is a phase contrast taken with an inverted microscope at 100× magnification. By taking at least 10 random images the ratio of beads with cells and average number of cells per bead was determined.

FIG. 2B is an image Jurkat cells attached to the larger magnetic agarose beads coated with CD3 antibody after 6 days incubation in the flow-through bioreactor. The image is a phase contrast taken with an inverted microscope at 100× magnification. By taking at least 10 random images the ratio of beads with cells and average number of cells per bead was determined.

FIG. 3A is an image of fibroblast cells grown on collagen coated beads in a test tube for 2 weeks. At the beginning of the 2^{nd} week the cells were exposed to FGF and after a week they grew a thin gel-like membrane over the beads (with some of the beads embedded in it). The membrane is shown here at 40× magnification.

FIG. 3B is an image of fibroblasts cells grown on collagen coated beads in a test tube for 2 weeks. At the beginning of the 2^{nd} week they were exposed to FGF and after a week they grew a thin gel-like membrane over the beads (with some of the beads embedded in it). The membrane is shown here at 100× magnification.

FIG. 3C is an image of fibroblasts cells grown on collagen coated beads in a test tube for 2 weeks. At the beginning of the 2^{nd} week they were exposed to FGF and after a week they grew a thin gel-like membrane over the beads (with some of the beads embedded in it). The membrane is shown here at 400× magnification.

FIG. 4A is an image showing histology staining of the fibroblast tissue grown in the test tube with a magnet. H&E staining (hematoxylin and eosin): In color, the cells and collagen are pink, while the nuclei are purple. The beads have their natural orange-brown color. The image was taken at 100× magnification.

FIG. 4B is an image of histology staining of the fibroblast tissue grown in the test tube with a magnet. With picrosirius red staining: in color, cells stain in green, and the collagen stains pink. The beads have their natural orange-brown color. The image was taken at 100× magnification.

FIG. 4C is photograph showing histology staining of the fibroblast tissue grown in the test tube with a magnet. The image was taken at 400× magnification.

FIG. 5A is an image showing histology staining of the smooth muscle cell tissue grown in the test tube with a magnet with H&E staining: cells and collagen stain in pink, while the nuclei stain in purple. The beads have their natural orange-brown color. The image was taken at 100× magnification.

FIG. 5B is an image showing histology staining of the smooth muscle cell tissue grown in the test tube with a magnet with in color picrosirius red staining. The image was taken at 100× magnification.

FIG. 6 is a schematic of a bioreactor and detailed schematic of annular magnet.

FIG. 7A is an image showing modified magnetic agarose beads (MABs) with collagen coating seeded with rat aortic smooth muscle cells (RASMCs).

FIG. 7B is an image showing modified MABs without collagen coating in rat aortic smooth muscle cells (RASMCs) suspension.

FIG. 7C is an image showing a close-up of modified MABs seeded with RASM cells.

FIG. 8A is a schematic of the MSCFB holding two separate RASM tissue rings.

FIG. 8B is an image of tissue rings growing in the MSCFB.

FIG. 9 is an image of RASM tissue growing in the MSCFB.

FIG. 10A is an image of RASM tissue taken out of the MSCFB.

FIG. 10B is an image of RASM tissue sections with and without MABs.

FIG. 11A is an image of RASM tissue section without MABs.

FIG. 11B is an image of a section of the RASM tissue tube transferred onto an agarose rod before embedding in paraffin and slicing it.

FIG. 12A is an image of the H&E stain of 29-day old tissue ring with MABs grown inside the magnetic field of the MSCFB. In color, the purple color shows the nuclei of the cells and the pink color shows the cytoplasm of the cells, and grey color shows the MABs.

FIG. 12B is an image of a section of the 29-day old RASM tissue tube without the MABs. This was the portion of the tissue tube grew from the tissue ring with MABs.

FIG. 12C is an image showing the close-up of the RASM tissue ring without the MABs grew from the tissue ring with MABs.

FIG. 13 is an image of Jurkat cells attached on the collagen coated rough MABs that had an irregular shape or tough surface.

### DETAILED DESCRIPTION OF THE INVENTION

A description of example embodiments of the invention follows.

The invention described herein pertains to bioreactor systems, systems for culturing cells and methods of utilizing a bioreactor for bioengineering methodologies such as tissue reconstruction, production of biofuel and bioproducts, production of cell products (e.g., generation of biologically produced pharmaceuticals) and cell amplification without the use of a solid support or scaffolding.

In certain embodiments, the use of magnetic beads and magnetic field allows for three dimensional manipulation of cell amplification into tissue and or the production of desired cellular products from cells. The methods and bioreactor are advantageous over existing method due to increased efficiency (e.g., costs and use of materials), minimal down time in running, harvesting and maintaining the system (e.g., due to the use of continuous feed). Additionally, in certain embodiments or methods of the invention, the need to have a support or scaffolding is avoided. In addition, cells attached to a magnetic support can be mechanically stimulated by varying the magnetic field, as shown by Dobson et al (Dobson, J, et al., NanoBioscience, IEEE Transactions, 5, 173-177 (2006), allowing for the replication of stress forces experienced by certain cell types, such as heart muscle cells. The magnetic field is varied to produce desired predetermined structure of the cells into tissue. The ability to vary the magnetic field allows for control of the growth and proliferation of the cells. The magnetically stabilized tissue in the bioreactor can be shaped to a desired structure by controlling the magnetic field and also the flow of the culture medium.

In a particular embodiment, a magnetically-stabilized, continuous-flow bioreactor (MSCFB) was designed and applied for the controlled growth of rat aortic smooth muscle cells (RASMC) in a pre-determined shape in a three-dimensional environment. The cells were immobilized on magnetic agarose beads (MABs) and grown into a tube-shaped tissue.

### DEFINITIONS

As used herein “magnetically stabilized continuous flow-through bioreactor” (MSCFB) refers to a fluidized bed of magnetic particles, such as iron, cobalt and their oxides or magnetite (and the like) that is stabilized by applying an external magnetic field.

As used herein, “magnetic beads” are beads made out of a magnetic material such as iron, cobalt and their oxides or magnetite (and the like). In certain embodiments, the beads are of a homogenous size or alternatively can be a variety of particle sizes. The beads can also be shaped for a desired cell or tissue morphology. The beads can be smooth, rough, geometrically shaped or irregular shaped. The beads are porous or alternatively non porous. The various properties of the beads allow for flexibility in the methods described. The beads should have no adverse effects on the cells. The bead are manipulated and controlled by a magnetic field and in certain embodiments by the continuous flow of fluid, for example, medium, to form a “template” or “pseudo scaffolding” allowing cells to culture without the detrimental effects of a solid scaffold and in a manner that mimics in vivo conditions. The beads can further be coated with a polymer, suitable polymers include agarose, PLA, degradable polymers and the like. These polymer surfaces can additionally be derivatized for attachment of other materials such as proteins, antibodies, or cytokines through chemical moieties. This further modification can be used for targeting the cells or tissue or interacting with other cells and tissue. In one embodiment, the magnetic beads along with another magnet form the magnetic field apparatus. Other apparatus that produce a magnetic field are also contemplated.

As used herein “three-dimensional culture” is a dynamic culture that allows for in vivo simulated growth for proper tissue functions. The cells and/or tissue are grown under conditions that mimic in vivo cellular conditions. The metabolic environment is adapted and externally controlled (e.g., via a magnetic field, fluidics and the like) to approach that of in vivo environment. This system allows cell to grow and interact in three dimensions in contrast to the two-dimensions of traditional cell culture systems. For example, the three dimensional culture could be used for the growth of heart tissue, that requires a dynamic environment for the pulsing cells to allow for movement and appropriate stress. Furthermore, tissue comprising two or more cell types is also contemplated. Such a two cell tissue culture and the use of a continuous fluid flow allows for the production of blood vessel like culture, where the lumen is generated by the continuous flow of fluid and the different cells are allowed to culture to produce the separate layers of the bioengineered vessel.

As used herein, “medium” or “culture medium” refers to a standard medium further supplemented with nutrients, cytokines, growth factors, hormones, salts or other molecules to generate a specialized growth and maintenance condition for each cell. In some aspects, the medium refers to a commercial medium. Other medium and additional nutrients may be supplemented and optimized as needed for control of cell growth and maintenance.

As used herein, “culture conditions” include conditions and concentrations that may contribute to the cell proliferation or maintenance, for example, conditions can include, inclusion of growth factors, and other nutrients, gas concentrations such as oxygen concentration (for example low oxygen conditions (about 1% to higher levels of about 10%), pH, pressures and temperature, flow conditions for producing the necessary mechanical properties for mimicking in vivo conditions. These conditions can be readily manipulated for enhancing and directing growth conditions. For example, the pH, pressure and temperature can be controlled to maximize cell growth.

As used herein, “adhesion molecules” refers to proteins and molecules located on the cell surface that while in the body are involved with the binding with other cells or with the extra-cellular matrix. In certain aspects for use with the reactor, system and methods described herein, the adhesion molecules facilitate the binding of a specific cell to the magnetic bead.

As used herein, a “bioreactor” is a device or system that supports a biologically active environment. For example, a bioreactor is a vessel operated under certain conditions for culturing organisms, cells or tissues. The system is maintained at a temperature of 37° C. and continuously monitored for pH, temperature and gas composition. Depending on the organism, the process is either aerobic or anaerobic. In certain embodiments, the bioreactor includes an apparatus for conveying fluids, thus providing a continuous feedback of medium enabling a continuous flow of medium through the cell culture from an external medium source. In certain embodiment, the vessel is stainless steel. In other embodiments, the vessel is made of a transparent material allowing interaction with light.

In other aspects, the bioreactor includes an apparatus for harvesting, for example to remove discarded cell products from the medium, for cell separations, or to identify and sort cell morphology. In other embodiments, the bioreactor includes an apparatus for dialysis. In still other embodiments, the bioreactor includes an apparatus for photoelectrochemical processing. Useful apparatus for performing these activities are known in the art and can be included with the bioreactor in the methods and systems of the invention.

As used herein, “operating conditions include temperature, pressure, liquid space velocity, gas, light, cell density, and magnetic field intensity and the like.

As used herein, “growth factor” refers to a molecule that has an effect on proliferation and maturation of cells and or tissue. Examples include but are not limited to PDGF, VEGF, EGF, FGFs, insulin, and other hormones or nutrients that assist in the proliferation, targeting and maturation of cells.

As used herein, a ‘tether” or “anchor” refers to when the linker and/or antibody molecule or other molecule that attaches the cell via a cell surface marker to the wall of the reactor, surface or other component of the compartment (such as a particle). Examples are described in Orsello et al., “Characterization of Cell Detachment from Hollow Fiber Membranes,” Biomed. Sci. Instrum., 35: 315-320 (1999) and Nordon et al. “Hollow-fiber assay for Ligand-mediated Cell Adhesion,” Cytometry A. 57:39-44 (2004).

As used herein, “derivatized polymer surfaces” refers to polymer based surfaces that have been chemically modified for attachment of chemical moieties, such as antibodies, adhesion factors and the like or modified so as to reduce un-desired or non-specific binding.

As used herein an “antibody” refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

Magnetically stabilized continuous flow bioreactors (MSCFB) are useful in tissue culture and pharmaceutical production. MSCFB allows the continuous collection of products from the cells stabilized in the magnetic field without the need to harvest the cells and re-seed the bioreactor. MSCFB also have potential applications in stem cell culture, tissue engineering and tissue regeneration. In conventional bioreactor mechanical shear is usually provided by stirring the culture medium, which will generate turbulent flow and may damage the growing tissue. In this design, no moving component except the laminar flow of culture medium is used and it provides the mechanical shear for the enhanced proliferation and maturation of tissue.

### Continuous Dialysis for Use in Fluidized Bed System

In certain embodiments, a “fluidized bed” is created in the bioreactor, the cells anchored or tethered via the cell surface marker to a surface, such as magnetic beads. In packed bed reactors that utilized lower fluid velocities, the cell substrates remain in place as the fluid passes through the voids in the material. As the fluid velocity is increased, the compartment will reach a stage where the force of the fluid on the cell material is enough to balance the weight of the material. Depending on the desired operating conditions and properties of each cell population various flow regimes may be observed in this reactor. In certain embodiments, the fluidized bed technology allows extreme cell densities while delivering nutrients, removing metabolites and waste and concentrating cytokines/growth factors.

Magnetic control of the entrapment particles (e.g., magnetic beads) will effectively create a dynamically controllable 3-D matrix.

Benefits of utilizing fluidized bed technology include:
Uniform Particle Mixing: Due to the intrinsic fluid-like behavior of the substrate, fluidized beds do not experience poor mixing as in packed beds. This complete mixing allows for a uniform product that can often be hard to achieve in other reactor designs. The elimination of radial and axial concentration gradients also allows for better fluid-solid contact, which is essential for reaction efficiency and quality.
Uniform Temperature and other Physical Parameter and Nutrient Gradients: A fluidized bed reduces temperature differences or concentration differences, especially hotspots, that can result in poor exposure of the substrate to the medium.
Ability to Operate Reactor in Continuous State: The fluidized bed nature of the compartments that comprise the reactor allows for the ability to continuously withdraw product or cell type and introduce new specialized medium into the particular compartments of the reaction vessel. Operating at a continuous process state allows the separation and differentiation of each cell type more efficiently due to the removal of startup conditions in traditional batch processes
In certain aspects, an advantage of intimate contact between cell and fresh fluid (compared to a barrier dialysis system) occurs when low dissolved O₂ concentrations are beneficial to the cells, for example, progenitor cells.

The terminal velocity of magnetic beads is on the order of about 10 μm/s. Thus, without an applied magnetic field, the particles would be entrained by the medium. The magnetic field enables the drag force of the fluid to be countered by the attractive magnetic force. In certain aspects, it is optimal to balance these forces to keep the particles separated and dispersed uniformly within the reactor.

In certain embodiments, aggregation between particles is minimized by controlling the surface charge on the bead surface, through SAM (self-assembled monolayer) technology to increase the surface charge of the beads, thereby opposing magnetic attraction with electrostatic repulsion. This approach creates a 3D net structure of magnetic particles with the inter-particle distance being tunable by adjusting the magnetic field at a given fluid velocity. The fluid velocity will be determined by nutrient and metabolite exchange requirements (for example, oxygen requirements). In certain aspects, overall volume may be determined by cell production capacity and achievable cell density for each cell type. The dimensional ratio of bed height to footprint may be determined by the fluid flow rate and properties of the magnetic net.

### Derivatized Polymer Surfaces

In another aspect, the invention provides for using various chemistries for attachment of any molecule(s), for example, antibodies, to a wide range of substrates to be utilized with the methods, system and bioreactor described herein. The surfaces or substrates include but are not limited to gold, stainless steel, indium tin oxide (ITO), glass, silicone, polystyrene, polycarbonate and many other polymers. Molecules for attachment useful in the present invention include hydrophobic and hydrophilic molecules, photolithographic molecules, chemical sensors, molecules with photoswitchable hydrophobicity as well as nucleic acids, peptides and proteins and the like.

A variety of methods for attaching (linking or conjugating) a cell or a ligand, such as an antibody to a surface can be used. In certain embodiments, linkers containing terminal functional groups are used to link to the surface. Generally, conjugation is accomplished by reacting the surface that contains a reactive functional group (or is modified to contain a reactive functional group) with a linker or directly with a ligand. Covalent bonds can be formed by reacting a surface that contains (or is modified to contain) a chemical moiety or functional group that can, under appropriate conditions, with a second chemical group thereby forming a covalent bond.

Many suitable reactive chemical group combinations are known in the art, for example, an amine group can react with an electrophilic group such as tosylate, mesylate, halo (chloro, bromo, fluoro, iodo), N-hydroxysuccinimidyl ester (NHS), and the like. Thiols can react with maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

The surface attachment of antibody to another molecule can be produced by reacting an appropriate ligand with a surface or molecule comprising a reactive chemical or functional group, as described herein. For example, conjugation may be accomplished via primary amine residues, carboxy groups and cysteine residues. Engineered cysteine residues provide certain advantages as sites for toxin conjugation, because the conjugation ligand via an un-paired cysteine residue (e.g., a cysteine residue engineered into a ligand) provides a method to achieve site specific conjugation and reduces the likelihood that the conjugation will interfere with antigen binding function. For example, the unpaired cysteine can be incorporated at the carboxy-terminus of an antibody ligand to provide a residue for site specific thiol conjugation. In addition, specific solvent accessible sites in ligand which are not naturally occurring cysteine residues can be mutated to a cysteine for attachment to the surface. Solvent accessible residues ligand can be determined using methods known in the art such as analysis of the crystal structures of a ligand.

Thiol conjugates can be prepared using any suitable method, such as the well-known methods for forming disulfide bonds or by reaction with a thiol reactive group such as maleimide, iodoacetyl, acrylolyl, pyridyl disulfides, 5-thiol-2-nitrobenzoic acid thiol (TNB-thiol), and the like.

A general procedure to attach the linker molecule to a polymer surface is to plasma clean the substrate for 10 minutes to maximize the number of hydroxyl groups on the surface. The surface is silanized by immersion, for no more than five minutes, in a 5 mM anhydrous ethanol solution of aminopropyl triethoxysilane. The surfaces are washed with ethanol and the molecule of choice is attached to the surface by formation of a peptide bond. The molecule to be attached, (containing an exposed carboxylic acid) is treated with N-hydroxysuccinimide (HSC) and 1,3-dicyclohexylcarbodiimide (DCC) in the presence of N,N-dimethyl aminopyridine (DMAP) as a base in the presence of the amine derivatized surface. The result is the coupling of the R group to the surface where R may be any of the molecular species mentioned earlier including antibodies.

The attachment linker or antibody forms a “tether” or anchor for attachment of the cell via the cell surface marker. Also contemplated herein is the attachment of two or more antibodies (same or different) to one linker. The number of tethers/cm² will be varied to deliver the optimum number for cell attachment. The optimum tether density is the minimum number required to bind the cell in the flow stream, while not interfering with cell maturation and eventual release of that cell upon down regulation of the tether marker.

In certain aspects, it is desirable that orientation of the antibody or linker molecule face the medium for contact with the cells, nutrients and other components of the fluids. Orientation can be accomplished using various methods known to one skilled in the art, for example, using the methods of Weiping et al. (Q. Weiping et al., J Colloid Interface Sci 214, 16 (1999)). Briefly, in the Weiping et al. method, the antibody or linker is oxidized in an aqueous solution of 50 mM sodium iodate at pH 5.2. Excess oxidant is removed using dialysis. The oxidized antibody or linker is then incubated with the amino terminated surface in the presence of 5 mM sodium borohydride in acetate buffer pH 5.2 for 12 hours. Orientation of the antibody or linker on these surfaces can been confirmed using atomic force microscopy. It is also possible to control the ordering of self assembled monolayers by the introduction of sterically bulky molecules such as tertiary butyl benzene and species with hydrophobic and hydrophilic moieties such as saturated alkane chains or polyols, into the molecular film. The introduction of such molecules controls the distribution of tethers across the substrate surface preventing clumping of the molecules into islands. Alternative attachment strategies include but are not limited to the following:
a) The use of a triethoxy silane linked with a 10 carbon chain to a protected carboxylic group to coat the cell culture surface. The carboxylic acid is protected since triethoxy silanes with free carboxylic groups are not stable. Upon irradiation with 355 nm light the protecting group is removed and the carboxylic group then is exposed on the surface. The antibody, linker or attachment molecule will be covalently immobilized on the carboxyl-derivatized surface. The carboxylic acid is reacted with N-(3-Dimethylaminopropyl)-N′-ethylcarbodiimide hydrochloride (EDC) in aqueous solution to form an unstable intermediate, an O-acylisourea. A solution of N-hydroxysuccinimide (NHS) reacts with this intermediate to form a metastable NHS-ester. This intermediate then undergoes nucleophilic substitution with a primary amino group on the antibody; and
b) The use of a maleimide derivative on the surface to attach the antibody. In this procedure the surface will be derivatized with amino groups using aminopropyl triethoxysilane. The amino terminated surface is treated with a 2% solution of Sulfosuccinimidyl 4-[N-maleimidomethyl]cyclohexane-1-carboxylate (SSMCC) in 50 mM phosphate buffer, pH 7.2 for 1 hour followed by washing with buffer. This reaction converts the amino terminal group to a maleimide. The antibody is then attached to the maleimide through reaction of sulfhydryl groups on the surface of the antibody. The maleimide derivatized surface is incubated in a 10 μM phosphate buffer solution of the antibody for 2 hours. The surface is then washed repeatedly with buffer to remove any physioadsorbed protein.

### Stability of Tether Coated Surfaces

Molecular tethers need appropriate stability to remain attached to magnetic surfaces for extended periods of time. For example, SAM chemistry shows that derivatized surfaces remain stable in phosphate buffered saline at 37° C. For flat samples of substrate material, the stability of the attached chemistry is assayed by grazing angle infra-red (IR) spectroscopy. Briefly spectra is acquired from derivatized substrates that have been maintained in conditions used in the bioreactor, i.e., flowing culture medium at 37° C. Surface spectra will be obtained with a Nexus FT-IR model 670 spectrometer equipped with a ThermoNicolet grazing angle accessory and a liquid-nitrogen cooled MCTA detector. The IR beam is incident at 75° angle on the substrate surface and the optical path is purged with nitrogen. Typically 64 scans are collected for each sample with a 4 cm⁻¹ resolution. The scan range is from about 4000 to about 1000/cm. A clean polymer substrate is used to acquire background spectra before and after data acquisition for each sample. For stability tests of chemically modified components of the bioreactor for which flat samples may not be available, the bioreactor will be run with cytokine supplemented CDM medium to simulate production conditions (37° C. and 5% O₂). Circulating medium will be sampled at defined intervals and the presence of released tethers quantified by ELISA (tether specific ligand assay). In alternative aspects, tether molecules can be co-localizing with molecules that minimize protein binding, such as short chain polyethylene glycols and the like.

### Tether Based Biosensor

A tethered biosensor can be utilized for the identification of specific cell surface molecules. For example, a biosensor using Lithium photosensor technology as described in Wanichacheva et al, 2006.

### Monitoring the Concentration of Magnetic Beads in the Fluidized Bed.

The concentration of magnetic beads at different positions within the fluidized bed reactor can be monitored by changes in the current required to maintain the net as beads move into the field. The presence of magnetic beads increases the inductance of the field coils around the reactor. This inductance change is sensed by a change in the current to the coils. Since the free beads and the bound beads position themselves at different levels within the reactor, it is possible to monitor the number of beads and the relative numbers of bound and unbound beads by using sensing coils at different positions within the reactor.

### Measurements

Gas concentration and nutrient concentration in the bioreactor can be controlled within the bioreactor.

### Spectroscopy

Laser diffraction and UV/Vis spectroscopy may both be implemented using optical fibers. These fibers may be coupled to their respective instruments using a fiber multiplexer, thereby minimizing the cost of measurements. Additionally, this system allows the use dual detector systems which allows for redundancy and improving the confidence in the measurements by ensuring the data from two instruments are coincident.

Laser diffraction (light scattering) may be used to determine particle size distribution within the bioreactor. Smaller cells scatter light the most and from the diffraction pattern, the distribution of cell sizes can be determined using Mie theory. The technique assumes that the cells are spherical and that they are dilute in the sample. The technique involves shining a laser beam through the cell population and imaging the diffraction pattern. In the final (maturation) stage of the bioreactor, the diffraction pattern will be monitored at up to 6 wavelengths. Five wavelengths from 516-592 nm are used to determine the HBO₂/Hb ratio and one in the near infrared serves as a reference.

### Biofuel Applications

One application of the Bioreactor and methodologies described herein is the development of a Biofuel bioreactor with both energy efficiency and cost efficiency. Specifically, a bioreactor for the energy-efficient production of lipid biomass with a measured energy efficiency ≧95% (EnergyIN/EnergyOUT) is described.

Bioreactor monitoring and functions includes identifying metabolic markers, correlating metabolism with fatty acid yield, using a continuous optical monitoring system (OMS), integrate pH, temp, gas, feedstock monitoring and a feedback system.

In certain embodiments, the measured energy efficiency is ≧80%, ≧85%, ≧90% and ≧91%, ≧92%, ≧93%, ≧94%, ≧96%, ≧97%.

In developing such a bioreactor, the liquid fuel type (diesel fuel, JP-8 aviation fuel, and/or high octane fuels for four-stroke internal combustion engines) is predetermined as well as the octane levels (e.g., liquid fuels ≧85 research octane or ≧40 octane are desirable). Further, liquid fuel energy density (e.g., ≧32 megajoules per kilogram); liquid fuel heat of vaporization (e.g., <0.5 megajoules per kilogram) and liquid fuel-energy-out to photon/electrical energy-in of the envisioned system; with an overall energy efficiency >1% should be anticipated. Additionally, amount and necessity of rare earth elements additives need to be determined.

A number of organisms are contemplated as systems to incorporate atmospheric CO₂ into organic molecules for the production of biofuels. These organisms include Rhodobacter sphaeroides, Synechococcus elongates, Phototrophic bacteria, Rdodopseudomanas rutiia, (an anoxygenic organism), Clostridiumm ljungdahlii (an anaerobic organism that utilizes organic waste and produces acetate and ethanol), Chlorogleopsis, (a thermophilic cyanobacterium) algae and the like. In its simplest form the bioreactor can accommodate a pure population of microorganisms. In this configuration the system provides as an immortal source of cells that provide the feedstock for the processing unit the final product of which is short chain alkanes. The organisms chosen may be anaerobic or aerobic and may also be photoactive. An entrapped population remains in the bioreactor, divides and yields daughter cells as a product.

The containment of the organism within the bioreactor is dependent on the organism shape and its surface chemistry. Attachment of both adherent and suspensions cells is possible using various surface attachment chemistries. Adhesion may be carried out through electrostatic interactions, antibody/antigen interactions or through chemical and photochemical crosslinking. Crosslinking of a number of organisms have shown that this chemistry may be carried out without adversely affecting cell viability.

The shape of the organism will to a certain extent determine the shape of the binding substrate in the bioreactor and the specific binding chemistry used to attach the organism. The binding substrate and chemistry will be chosen to maintain the viability of the entrapped organism and maximize the concentration of undeterred daughter cells from the reactor. Spherical bacteria that undergo division along three planes (Staphylococci) tend to form clusters of cells. For these organisms small beads will be used to entrap the cells minimizing subsequent binding of daughter cells. Bacteria that undergo division along one plane (Bacillus) will be attached to substrate at one end of the organism.

In other embodiments, more complex bioreactor systems are include one bioreactor stage feeding a second stage reactor housing a secondary organism maintained under separate conditions. In this way both anaerobic and aerobic cultures may act on waste water producing reducing equivalents and fatty acids. Dialysis and gas exchange systems in the flow of the two-stage system provide optimum conditions and allow recycling of feedstock to maximize utilization of the substrate. The organism to biofuel conversion (OBF) is determined and optimized.

The biofuel bioreactor described here can be used to culture a number of different cell types and is scalable to produce large quantities of cells at concentrations that would not be sustainable in a conventional batch reactor or in an open pond.

An advantage of the biofuel bioreactor over pond system biofuel production is that true anaerobic organisms may be grown under carefully controlled concentrations of oxygen, carbon dioxide, nitrogen and other gases. See Energy biotechnology with cyanobacteria by S A. Angermayr, et al., (Current Opinion in Biotechnology (2009) 20, 257-263) and Direct photosynthetic recycling of carbon dioxide to isobutyraldehyde by S. et al., (Nature Biotechnology (2009) 27(12), 1177-1182) for descriptions of possible organisms.

The system described here utilizes a range of surface chemistries to attach the biofuel organisms to beads. These include specific attachment using antibodies and non specific attachment using collagen, polysine and collagen analogues and the like. It is also possible to use the biofuel bioreactor with surfaces to which organisms will not attach.

To maintain adherence to a critical timeline the system will initially use the organism Synechococcus elongates. A cyanobacterium that may survive under a wide variety of conditions including saltwater, freshwater and a wide range of temperatures is also contemplated.

Synthesized Magnetic Bead with Attachment Chemistry

Magnetic beads of various sizes and chemistry for attachment can be used in the methods and apparatus described herein. Agarose magnetic beads are synthesized according to a modified procedure described in more detail in the Examples. Briefly iron oxide nanoparticles are prepared by precipitation from a dodecanoic acid solution with ammonium hydroxide while rapidly stirring. The magnetic agarose beads are formed by the co-emulsification of a hot, aqueous, agarose solution with iron oxide particles in an oil bath. The size of the beads is dependent on the stirring rate, the temperature and the relative ratio of the aqueous solution volume to the oil volume. Beads obtained by this method vary in size from 1 μm to 100 μm, for example 100 nanometers, 100 nm to 500 nm, 500 nm to 1 micron, 750 nm to 10 microns, 10 micron to 100 microns, and ranges in between. In certain aspects, these bead sizes are be graded by centrifugation. Smaller particles by direct derivatization of the nanoparticles using self assembled monolayer technology are also available for use.

In producing the particles, 10 mL of magnetic fluid and the mixture is stirred vigorously with a glass stirring rod, while keeping it in the 90° C. water bath. Using a 3 mL syringe and an 18G needle, the magnetic mixture is immediately transferred dropwise to the oil solution while stirring at 600 rpm with the overhead mixer (the syringe is kept warm by squeezing the magnetic mixture in and out of the beaker a few times prior to transferring it to the oil mixture). After all the magnetic mixture is added the emulsion is stirred for an additional 10 min at 90° C. The water bath is cooled down by carefully removing most of the hot water and adding ice to it until the temperature drops to at least 15° C. and the emulsion is stirred for another 20 min. The magnetic agarose beads that are formed after the cooling are recovered from the oil phase by adding acetone to the emulsion mixture until it becomes clear, and placing it over a magnet. The magnetic beads are rinsed several times with acetone to remove any soybean oil and at the end they were thoroughly rinsed with deionized water. The magnetic beads can be stored in DI water at 4° C.

### Chemical Modification of Magnetic Beads

The magnetic beads (e.g., magnetic agarose beads) are modified for increasing the surface chemistry (e.g., concentration of carboxyl groups). Briefly magnetic beads are suspended in a freshly prepared 1 M chloroacetic acid solution in 3 M NaOH. The suspension is stirred for 70 min on an orbital shaker at room temperature. The reaction is stopped by adding 4 mg/mL of solid NaH₂PO₄ to the solution and then adjusting the pH to neutral with 5 M HCl. The magnetic beads are recovered over a magnet and rinsed with DI water.

A variety of proteins and other chemicals can then be attached to the beads through chemical modification. For example, antibodies can be attached to the surface. To increase the flexibility of antibodies that may be attached to the surface, a PEG spacer is covalently attached to the carboxyl groups prior to antibody attachment. In one such protocol, magnetic beads are transferred in a 1.5 mL centrifuge tube and are suspended in a small volume (˜0.5 mL) of freshly prepared solution of 100 mM EDC and 20 mM NHS in DI water. The suspension is agitated on an orbital shaker for 15 min at room temperature. The beads are rinsed once with DI water. This is done either by collecting the beads over a magnet or spinning the tube on a bench top centrifuge. A 100 μL solution of 100 mM NH₂—PEG₄₀-COOH in pH 9.6 carbonate buffer is prepared and added to the magnetic beads. The suspension is incubated overnight at room temperature and then it was rinsed three times with DI water.

In some embodiments of the invention, attachment of organism to the magnetic bead is useful. The attachment is accomplished by different surface chemistries for attaching a wide range of biological materials including organism to surfaces. These chemistries include but are not limited to passive interactions, active interactions (involving ligand binding mechanisms) and covalent linkages. Certain parameters are determined such as determining longevity of a cell or organism (e.g., microbe) on bead, determining free cell (organism, microbe) yield and the minimum medium/cell (organism, microbe) ratio.

Coatings for use on the surface of the magnetic beads include those that promote passive attachment of a cell (microbe, organism) include collagen, polylysine and short chain and small molecule analogs of these materials. These functionalities are applied to virtually any substrate, including metals and non-metals.

For example, active interactions have exploited the specific ligand binding properties of antibodies with cell surface markers, e.g, antibodies for stem cell surface markers demonstrated using surface Plasmon resonance. Covalent linkages are carried out using both photochemically initiated crosslinking and chemical crosslinking through the formation of a peptide bond. Photochemical crosslinking of cells is carried out to mammalian cells without disturbing the proper cell function.

### Model Fluid Dynamics as a Function of Bead Shape and Size

Optimization of the bioreactor flow conditions is done for maximizing the cell concentration to the amount of medium present. A number of parameters are optimized to ensure the optimum conditions for the reactor. The principle factors for optimization are: magnetic bead size, electrostatic interaction of the beads, bead shape, cell or organism (microbe population) size, medium flow rate and magnetic field strength.

Determining the optimum set of conditions is a dynamic problem that changes following the initiation of the bioreactor system and the modification of the conditions as the reactor reaches its mature operating condition. Any perturbation of these conditions requires a precise intervention to prevent the reactor becoming chaotic and shutting down. Furthermore, by determining correlations between conditions and biofuel production, the efficiency can be increased

### Monitoring the Concentration of Magnetic Beads in the Fluidized Bed.

The concentration of magnetic beads at different positions within the fluidized bed reactor is monitored by changes in the current required to maintain the net as beads move into the field. The presence of beads magnetic beads increases the inductance of the field coils around the reactor. This inductance change is sensed by a change in the current to the coils. Since the free beads and the bound beads position themselves at different levels within the reactor, it is possible to monitor the number of beads and the relative numbers of bound and unbound beads by using sensing coils at different positions within the reactor.

In certain aspects, an advantage of intimate contact between cell and fresh fluid (compared to a barrier dialysis system) occurs when low dissolved O₂ concentrations are beneficial to the cells, for example, progenitor cells.

In other aspects for cell capture, the magnitude of the spaces between the magnetic particles (1-4 μm in diameter) and the cells should be on the order of the cell size for example about 30 μm for CD34+HSCs and vary according to mitotic status and maturation stage. Use of a magnetic field may provide considerable operational flexibility. For example, feeding and harvesting cells could be performed at intermittent intervals with altered field strength to improve mixing and therefore cell-particle adhesion. Another advantage is that as the cells mature and detach from the beads, the effective radius of the beads will change dramatically. The ordering of magnetic beads in a flow stream at the center of a solenoid has previously been demonstrated. See Garcia, SCCSD et al., American Society of mechanical Engineers International mechanical Engineering congress and Expositions, NY, N.Y. (Nov. 11-16, 2001)). The establishment of a uniform radial profile of magnetic particles using an applied magnetic field in a 2.5 mm diameter bed has also been demonstrated by Burns and Graves, Chemical Engineering Communications 67Z:315 (1998)), albeit for much larger particles (100 μm).

Determination of useful parameters for particle size, surface charge and magnetic field strength should be balanced with the parameters and conditions for the entrapment and maturation of cells.

### Method and Applications

The methods and bioreactor described herein can be used for rowing 3D tissue in the magnetically-stabilized, continuous-flow bioreactor (MSCFB).

This technology demonstrates the potential of using this bioreactor for the generation of tissue in a desired shape and also continuous production of cell metabolites. This bioreactor system will also easily allow for the addition of other cell types or layers resulting in the generation of more complex tissues. Using external control of the tissue shape by modifying the magnetic field, it is possible to generate tissues of various cell types and potentially organs. The continuous production of cell products including (cells themselves) removes the need for harvesting and re-seeding of bioreactors, a disadvantage of traditional culturing methodologies. Additionally, the methods can be used to generate biofuels from microorganisms and other desirable cellular products, including pharmaceuticals and cell metabolites.

### EXEMPLIFICATION

### Example 1

### Preparation of Magnetic Fluid

Dodecanoic acid (2 g) was added to a 200 mL aqueous solution of 0.12 M ferrous chloride and 0.24 M ferric chloride in a 600 mL beaker. After adding slowly 40 mL of 25% ammonia solution, the mixture was placed in a water bath at 50° C. and stirred at 1300 rpm with an overhead mixer from G. K. Heller Corporation (Floral Park, N.Y.). The process was allowed to run for 30 min, while removing the lather that was continuously formed. The precipitate was collected over a magnet and then rinsed with 0.5% ammonia aqueous solution several times. A 100 mL volume of 1 g/L dodecanoic acid suspension in DI water was transferred to the precipitate and the mixture was stirred at 1300 rpm in the water bath at 80° C. for 30 min. The magnetic fluid that was formed was stored at room temperature in a sealed container shielded from light until further use.

### Preparation of Agarose Magnetic Beads

The beads were prepared by emulsification. A 160 mL soybean oil solution containing 5 g of polysorbate was added to a 600 mL beaker. The solution was stirred at 600 rpm with the overhead mixer and heated to 90° C. in a water bath. At the same time 10 mL of magnetic fluid were heated to 90° C. in the water bath, while 0.40 g of Seakem LE agarose were dissolved in 10 mL of DI water by heating the mixture for 50 seconds in a microwave oven. The hot agarose solution was immediately added to the heated 10 mL of magnetic fluid and the mixture was stirred vigorously with a glass stirring rod, while keeping it in the 90° C. water bath. Using a 3.0 mL syringe and an 18G needle, the mixture was added drop-wise to the soy bean oil solution while stirring at 600 rpm with the overhead mixer. The emulsion was stirred for 10 min at 90° C. The water bath was cooled down by adding some ice to it and the emulsion was stirred for 20 min at approximately 15° C. The magnetic agarose beads that were formed after the cooling were recovered from the oil phase by placing the emulsion over a magnet. The collected magnetic beads were rinsed several times with acetone to remove the soybean oil and at the end they were thoroughly rinsed with deionized water. The magnetic beads were stored in DI water at 4° C. until further use.

### Chemical Modification of Magnetic Beads

The magnetic agarose beads underwent a modification step in order to increase the surface concentration of carboxyl groups. The magnetic beads were suspended in a freshly prepared 1 M chloroacetic acid solution in 3 M NaOH. The suspension was stirred for 70 min on an orbital shaker at room temperature. The reaction was then stopped by adding 4 mg/mL of solid NaH₂PO₄ to the solution and then adjusting the pH to neutral with 5 M HCl. The magnetic beads were recovered over a magnet and rinsed with DI water.

Coating of beads with protein was done by EDC/NHS activation. Prior to the activation step the magnetic agarose beads were kept in a 70% ethanol solution in DI water for 1 hr in order to make them sterile. The DI water and all the solutions used after this step were sterile or were filtered through a 0.2 μm HT Tiffryn membrane filter to sterilize them. The sterile beads were rinsed three times with DI water. A fresh solution of 100 mM EDC and 20 mM NHS in DI water was prepared and 0.5 mL was added to the magnetic beads. The suspension was allowed to sit at room temperature for 15 min. The beads were rinsed once with DI water. Subsequently, a 100 μL solution of 100 μg/mL of antibody in pH 9.6 buffer solution or 1 mg/mL of collagen in pH 7.4 buffer was prepared and added to the magnetic beads. The solution was incubated overnight at 4° C. The next day the beads were rinsed with PBS, three times and stored at 4° C.

### Construction of Continuous Flow-Through Magnetic Bioreactor

A continuous flow bioreactor was constructed to culture three dimensional tissue stabilized by magnetic field in the solution medium. The continuous flow and suspension in the solution provides the mechanical stress factor as well as freedom required for enhanced proliferation of cells seeded on the magnetic beads. In addition to culturing cells and tissue, this bioreactor has high potential for a variety of industrial applications such as production of biofuels, pharmaceuticals, and cellular metabolites.

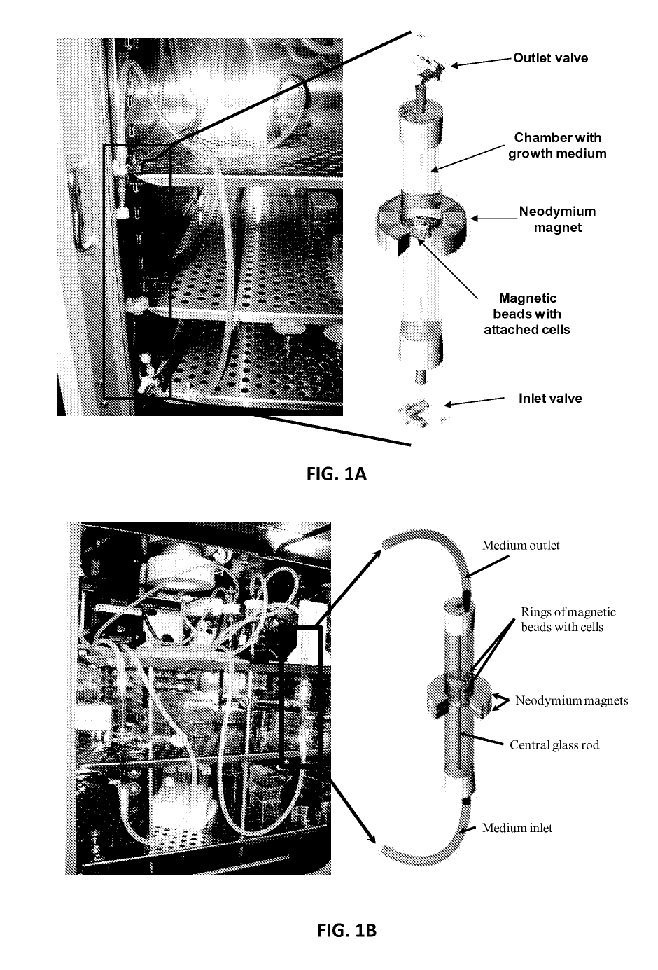

The flow-through bioreactor contains the following components: the magnetic bioreactor system (see FIG. 1A), the medium reservoir, the pump, the silicone conduit. The cells were seeded on the chemically modified magnetic beads stabilized in the magnetic field while the nutrient required for the cells was continuously provided by the flow of medium facilitated by peristaltic pump. The bioreactor and the reservoir were placed in a humidified 37° C. with 5% CO₂ incubator for optimum cell growth and the pump is placed outside of the incubator.

### Specifications

8 mm dia.×180 mm borosilicate glass tubing, 10 mm white rubber septa, 16 gauge hypodermal needles, Peristaltic pump, 3 way control valves, 5 mm Silicone tubing, Neodymium magnet, T25 BD plastic tissue culture flask, 10 ml test tube

### Results

Assembly of a continuous flow-through magnetic bioreactor system and CAD drawing of continuous flow-through magnetic bioreactor system is shown in FIG. 1A. The continuous flow-through magnetic bioreactor system assembled in the incubator (left) and CAD drawing of continuous flow-through magnetic bioreactor (right). The peristaltic pump generating the flow in the silicone tubing sits outside of the incubator. The source of the growth of the medium is a tissue culture flask that it is sitting flat in order to allow for gas exchange of the growth medium.

### Cell Culture

### Growing Jurkat Cells in the Flow-Through Bioreactor

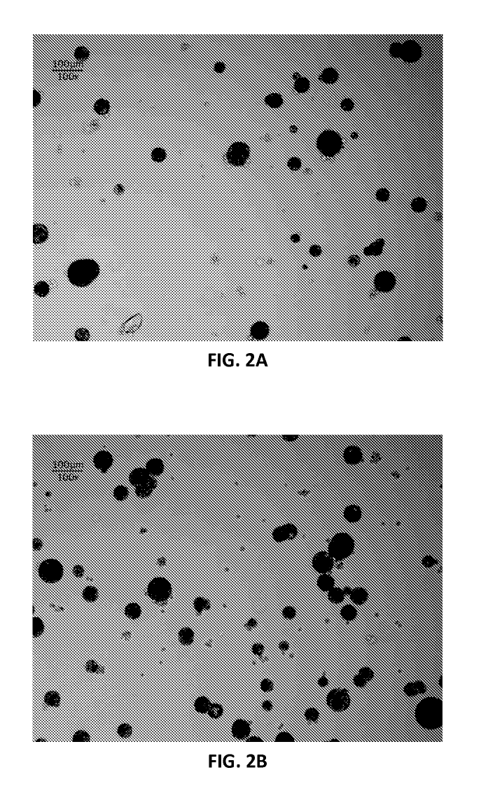

Jurkat cells are derived from lymphoma (cancerous white blood cells) and grow in suspension. They were grown in RPMI medium supplemented with 10% fetal bovine serum (FBS) and 1% penicillin/streptomycin were maintained in the incubator at 37° C. and 5% CO2. For cell attachment experiments the cells were dispersed by pipetting aseptically, as they tend to form small aggregates during their growth, and then the cells were counted, diluted in growth medium and exposed to magnetic agarose beads at a concentration of 1×105 cells/mL in a tissue culture flask. The beads had been coated with an antibody that binds specifically to an antigen (CD3) expressed in Jurkat cells. The cell and bead suspension was incubated overnight in the flask to allow the cells to attach to the beads (see FIG. 2A). The flow-through bioreactor system was flushed with 70% ethanol to sterilize it and then the ethanol was rinsed three times with PBS. The jurkat cell/magnetic bead suspension was transferred in the bioreactor chamber. Samples were taken from the bioreactor at different days using a needle attached to a plastic syringe. Cells stayed attached to the beads for at least 6 days (see FIG. 2B). After taking at least 15 random images the average ratio of beads with attached cells and the average number of cells per bead was determine. The results are given in Table 1.

Results

As shown in FIG. 2A, Jurkat cells are attached to the larger magnetic agarose beads coated with CD3 antibody after the overnight incubation. The image is a phase contrast was taken with an inverted microscope at 100× magnification. By taking at least 10 random images the ratio of beads with cells and average number of cells per bead was determined.

In FIG. 2B, Jurkat cells attached to the larger magnetic agarose beads coated with CD3 antibody after 6 days incubation in the flow-through bioreactor. The image is a phase contrast was taken with an inverted microscope at 100× magnification. By taking at least 10 random images the ratio of beads with cells and average number of cells per bead was determined.

Table 1. Jurkat cells in the flow-through bioreactor. The average ratio of beads with attached cells and average number of cells per bead was determined. The average values were obtained after counting the beads and cells in at least 10 random images for each time point.

TABLE 1

| | % beads | Average number |
|---|---|---|
| day | with cells | of beads per cell |
| 1 | 47.9 | 2.2 |
| 4 | 70.4 | 3.3 |
| 6 | 65.4 | 3.3 |
| 8 | 21.4 | 1.9 |

### Example 2

### Growing Fibroblasts with Magnetic Beads in a Glass Tube

Mouse neo-natal fibroblasts (CRL-2097) were grown in DMEM/F12 medium supplemented with 10% fetal bovine serum (FBS) in an incubator at 37° C. and 5% CO₂. Cells were first allowed to proliferate in the culture flasks. The cells were suspended in growth medium solution containing magnetic beads coated with collagen and then the mixture was transferred into a 10 mm diameter glass tube which had been coated with poly9-ethylene glycol) (PEG) to prevent non-specific adhesion of proteins and fibroblasts. A magnet was placed around the tube to create a ring of magnetic beads. Therefore if cells were attached to the cells they would form a ring as well after producing extra cellular matrix. The tubes with the magnet were kept in the 37° C. incubator and the medium was changed every 2 days. After 1 week the medium was replaced with DMEM/F12 with 10% FBS containing 4 ng/ml fibroblast growth factor (FGF), and the medium was replaced every 3 days. After 1 week the cells formed a membrane that appeared to follow the shape of the magnetic beads.

### Results

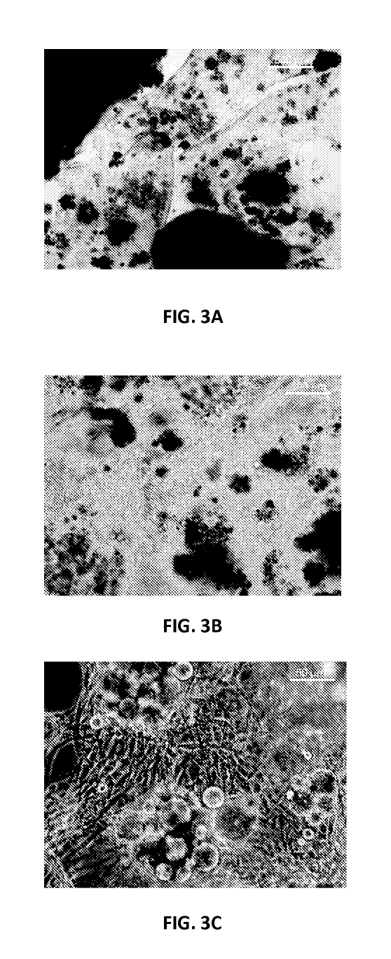

Fibroblasts cells grown on collagen coated beads in a test tube for 2 weeks. At the beginning of the 2nd week they were exposed to FGF and after a week they grew a thin gel-like membrane over the beads (with some of the beads embedded in it). The membrane is shown in FIG. 3A. at 40× magnification.

Fibroblasts cells grown on collagen coated beads in a test tube for 2 weeks. At the beginning of the 2nd week they were exposed to FGF and after a week they grew a thin gel-like membrane over the beads (with some of the beads embedded in it). The membrane is shown in FIG. 3B at 100× magnification.

Fibroblasts cells grown on collagen coated beads in a test tube for 2 weeks. At the beginning of the 2nd week they were exposed to FGF and after a week they grew a thin gel-like membrane over the beads (with some of the beads embedded in it). The membrane is shown in FIG. 3C at 400× magnification.

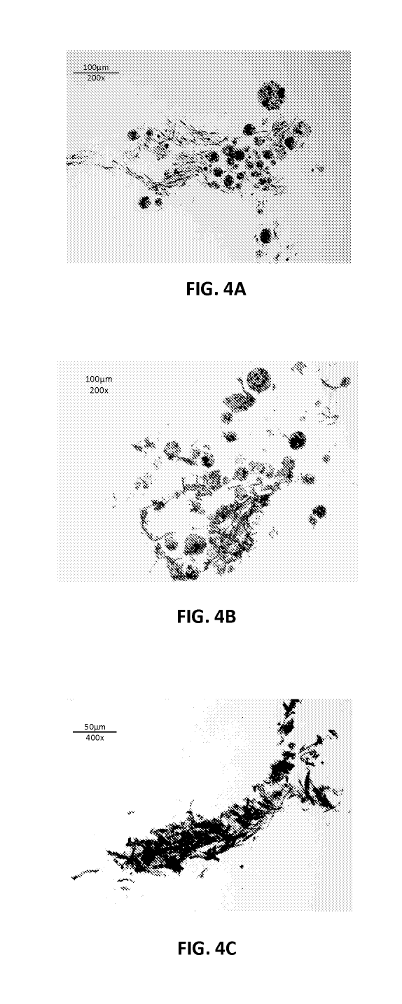

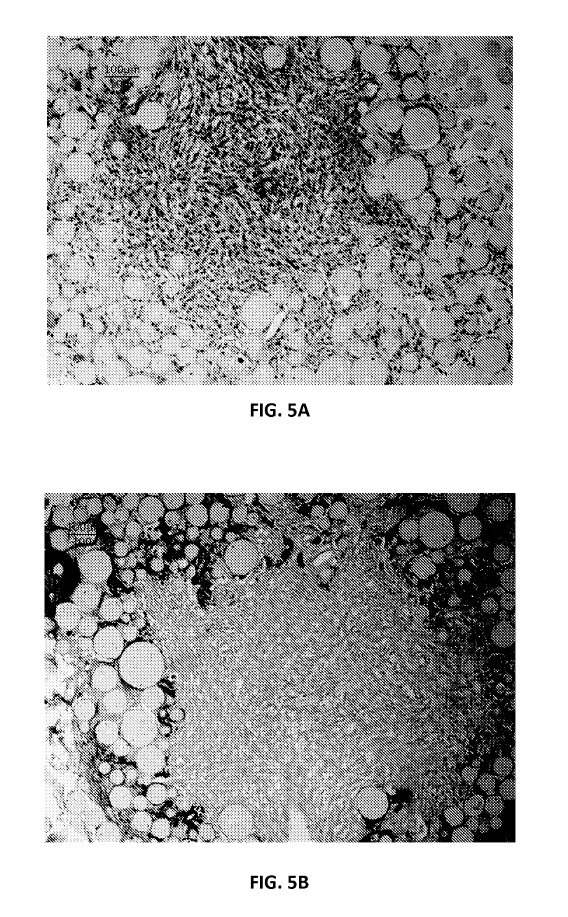

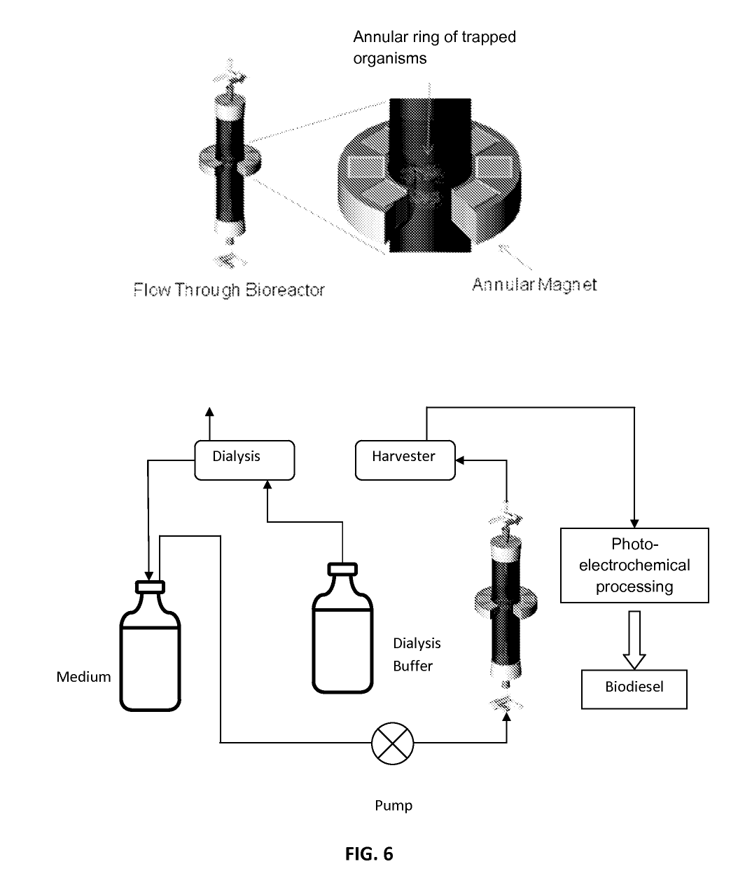

The membranous tissue grown over the beads was stained to confirm the presence of the cells and the collagen produced as part of the extra cellular matrix (see FIGS. 4A-4C).

### Example 3

### Growing Smooth Muscle Cells with Magnetic Beads in a Glass Tube

Rat aortic smooth muscle cells (RASMC) were grown in DMEM supplement with 10% FBS and 1% penicillin/streptomycin in 37° C. and 5% CO₂ incubator. Agarose magnetic beads were activated and coated with collagen as mentioned above in the magnetic bead section. The collagen coated magnetic beads were finally rinsed with PBS and resuspended in the 0.5 ml of cell culture medium, containing 1 ml of DMEM+10% FBS+1% pen/strep. The 25 mm diameter×20 mm neodymium magnet was secured around the top edge of the 5 ml borosilicate glass tube. The bottom end of the PEG coated bioreactor (glass tubing of 5 mm diameter×100 mm) was sealed with rubber septum while leaving the top open, and it was inserted into the test tube to position the magnet around the bioreactor. About 1 ml of culture medium was added into the bioreactor, and then, the magnetic beads solution was slowly pipette into the bioreactor from the top so that the beads are suspended above the magnet completely covering the cross section of the reactor tube. Finally, 0.5 ml of RASMC (5×105 cells) solution was added onto the beads and the bioreactor was incubated at 37° C. and 5% CO2. After 3 days, it was observed that the tissue was formed based on the geometry of the content in the bioreactor, and so it was taken out of the reactor and the histological analysis was performed on the tissue (see FIGS. 5A and 5B). By closing the top end with another septum, this magnetic bioreactor can be easily introduced to the continuous flow-through system to provide the mechanical stress factor and continuous gas exchange to enhance the tissue growth in a longer period.

### Example 4

### Materials

The following chemicals were purchased from Alfa Aesar, Ward Hill, Mass.: dodecanoic acid (Lauric acid) 98%, iron(II) chloride tetrahydrate 98%, iron(III) chloride hexahydrate 97.0-102.0%, N-hydroxysuccinimide 98+% (NHS), 1-(3-dimethylaminopropyl)-3-ethylcarbodiimide hydrochloride (EDC) 98+%, ferrous chloride (Iron (II) chloride tetrahydrate, 98%), ferric chloride (iron (III) chloride hexahydrate, 97-102%), ammonia, succinic anhydride, chloroacetic acid, 99+%, EDC (1-(3-dimethylaminopropyl)-3-ethylcarbodiimide hydrochloride), NHS(N-Hydroxysuccinimide, 98+%). Rat aortic smooth muscle cells (RASMC) were derived from smooth muscle cells isolated by enzymatic digestion of 3 month old adult male Wistar-Kyoto rat aortas (WKY3M-22 [Lemire et al., 1994; Lemire et al. 1996]). RASMC were cultured in DMEM (1×, Mediatech Inc., Manassas, Va.) supplemented with 10% FBS (PAA) 4.5 g/l glucose, L-glutamine and sodium pyruvate, and RPMI 1640, 1× supplemented with L-Glutamine and 1% penicillin/streptomycin were also from Mediatech, Inc. JT Baker brand was used for polysorbate 80 (Mallinckrodt Baker, Inc., Phillipsburg N.J.). SeaKem® LE brand was used for agarose (Lonza Group Ltd, Switzerland). Spectrum brand was used for soybean oil. (The Hain Celestial Group, Inc., Boulder, Colo.) The saline buffer solution (pH 9.6) was prepared in the lab. PureCol® brand was used for ultrapure bovine collagen type I (Advanced BioMatrix, Inc., San Diego, Calif.). Sodium dihydrogen phosphate monohydrate, 98+%, sodium azide, and PEG (Tryengylene glycol mono-11-mercaptoundecyl ether, 95%) were purchased from Aldrich Chemical Company, Inc., Milwaukee, Wis. Mouse monoclonal antibody to human CD3, DIO cell membrane fluorescent dye, and human IL-2 immunoassay kit were purchased from Invitrogen Co., Camarillo, Calif. Human IL-2 ELISA kit was also purchased from eBioscience, Inc., San Diego, Calif. EMD brand was used for sodium hydroxide (EM Science, Gibbstown, N.J.). PrecisionGlide® was used for hypodermic needle (Becton Dickinson & Co., Franklin Lakes, N.J.). Septa for culture medium reservoirs were from Ace Glass, Inc., Vineland, N.J., and those for bioreactors were bought from Sigma Aldrich, St. Louis, Mo. Borosilicate glass tubes, silicone tubing (0.125 ID×0.25 OD×0.063 wall), and ball type flow Indicator were purchased from VWR International, West Chester, Pa. MasterFlex® brand peristaltic pump (model 7553-60), and 4-way male slip stopcock were purchased from Cole-Parmer, Vernon Hills, Ill. Orbital shaker (model 1314) was from Barnstead/Lab-Line, Melrose Park, Ill. Heavy duty laboratory stirrer was from GK Heller Corp., Floral Park, N.Y. Steri-Cycle CO₂ incubator was purchased from Thermo Form a, Marietta, Ohio. Acrodisc syringe filter was ordered from Pall Life Sciences, Ann Arbor, Mich. Jurkat cells (clone E6-1) derived from human T lymphocyte were bought from ATCC (Manassas, Va.).

### Preparation of Magnetic Fluid

Dodecanoic acid (2 g) was added to a 200 mL aqueous solution of 0.12 M ferrous chloride and 0.24 M ferric chloride in a 600 mL beaker. The mixture was placed in a water bath at 50° C. and stirred at 1300 rpm with an overhead mixer from G. K. Heller Corporation (Floral Park, N.Y.). After adding slowly 40 mL of 25% ammonia solution, the suspension was kept at 50° C. and stirred continuously at 1300 rpm. The process was allowed to run for 30 min, while removing any lather that was formed. Thereafter, the precipitate was recovered from the suspension by placing the beaker over a magnet and it was rinsed several times with 0.5% ammonia solution. The precipitate was then transferred into a 100 mL suspension of 1.0 g/L lauric acid (dodecanoic acid) in DI water (the acid was crushed into a fine powder prior to making the suspension). The suspension was heated to 80° C. and stirred continuously at 1300 rpm for 30 min. The magnetic fluid that was formed was left overnight to settle, and most of the clear liquid layer was then removed with a pipette. The magnetic fluid was stored at room temperature in a sealed container shielded from light until further use.

### Preparation of Magnetic Agarose Beads (MABs)

A 10 mL volume of magnetic fluid was diluted with 10 mL of DI water, brought to pH 7.0 with 0.5% ammonia solution, and then sonicated for 30 min. A 180 mL soybean oil solution containing 30 g/L of polysorbate was prepared in a 400 mL beaker. The oil solution was stirred at 630 rpm with the overhead mixer and heated to 95° C. in a water bath, while making sure that the stirrer is submerged deep enough so that it doesn't create bubbles. At the same time 20 mL of the magnetic fluid prepared above were heated to 95° C. in the water bath, while a 10 mL aqueous solution of 4% Seakem LE agarose was prepared in a 150 mL beaker by heating for 50 sec in a microwave oven (making sure all the agarose was dissolved). The hot agarose solution was immediately added to the heated 20 mL of magnetic fluid and the mixture was stirred vigorously with a glass stirring rod, while keeping it in the 95° C. water bath. Using a 3 mL syringe and an 18G needle, the magnetic mixture was immediately transferred drop wise to the oil solution while stirring at 600 rpm with the overhead mixer (the syringe was kept warm by squeezing the magnetic mixture in and out of the beaker a few times prior to transferring it to the oil mixture). After all the magnetic mixture was added the emulsion was stirred for an additional 10 min at 90° C. The water bath was cooled down by carefully removing most of the hot water and adding ice to it until the temperature dropped to at least 15° C. and the emulsion was stirred for another 20 min. The MABs that were formed after the cooling were recovered from the oil phase by adding acetone to the emulsion mixture until it became clear, and placing it over a magnet. The magnetic beads were rinsed several times with acetone to remove any soybean oil and at the end they were thoroughly rinsed with deionized water. The magnetic beads were stored in DI water at 4° C. until further use, or 0.1% w/v of sodium azide was added for long term storage.

### Activation of MABs

Agarose is composed of polysaccharides and therefore the surface is covered with hydroxyl groups. Activation of hydroxyl groups for protein attachment requires the use of organic chemicals that could damage the beads and be toxic to the cells, so the hydroxyls (OH) groups were modified to carboxyl (COOH) groups with a simple reaction that occurs in aqueous solutions. When hydroxyls are exposed to chloroacetic acid (1M) in the presence of a base catalyst (3M), the oxygen of the hydroxyl binds to the α-carbon by replacing the chlorine atom. Activation of the COOH groups for protein attachment can then be done in aqueous solutions without affecting the beads or the cells. Modification of hydroxyl groups to carboxyl groups on magnetic agarose beads is shown below.

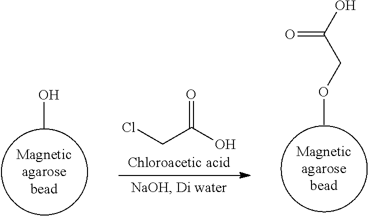

### Immobilization of Collagen on Magnetic Agarose Beads Via EDC-NHS Coupling. (See Below).

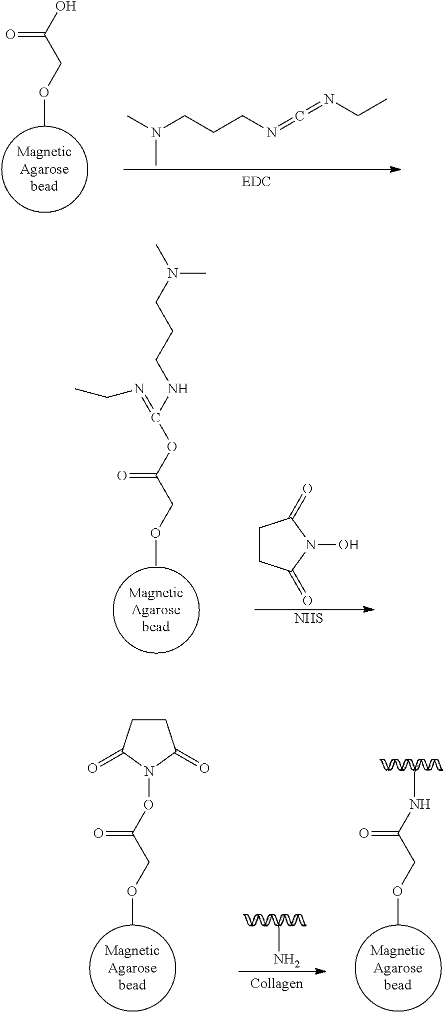

Prior to the activation step 0.02 mL MABs were transferred to each 1.5 mL Eppendorf tube and sterilized by incubation in 0.5 mL of 70% ethanol for 15 min at room temperature using the vertical rotator. The deionized water and all the solutions used after this step were sterile or were sterilized by autoclaving and filtering through a 0.2 μm HT Tiffryn membrane filter from Pall Life Sciences (Port Washington, N.Y.). The sterilized beads were rinsed three times with autoclaved filtered deionized water, and then collagen was immobilized on the surface of the beads via EDC-NHS coupling as described. A fresh solution of 100 mM EDC and 20 mM NHS in DI water was prepared and 0.5 mL was added to the magnetic beads via a 0.2 μm syringe filter. The suspension was incubated at room temperature in vertical rotation for 20 min. The beads were rinsed once with DI water. Subsequently, 0.4 mL of 1 mg/mL collagen in DI water solution was added to each centrifuge tube with magnetic beads. The centrifuge tubes with beads and collagen solution were rotated on the Mini-Pump Variable Flow for 2 hours and then incubated overnight at 4° C. Afterwards, the beads were rinsed twice with water and once with the appropriate culture medium (Cellgro DMEM 1×+10% FBS+1% Pen/Strep) before seeding the cells on the beads.

### Cells Adhesion to MABs

Magnetic beads, coated with collagen, were put into the 17×100 mm polystyrene tubes with dual position caps. The cap was tightly closed and a 1.0 μm syringe filter was inserted into a previously created hole on the cap to allow effective gas exchange. Then, 2 ml of culture medium with RASM cell concentration of 2×10⁵-4×10⁵ cells/mL was added in each tube, and the tubes were rotated horizontally for 9-12 hours. After that time, the tubes were taken off the rotator, the cell-seeded beads were collected on the bottom of the tubes by the magnet and after aspirating the excess cell solution, and the beads were resuspended in 1 ml of 37° C. pre-warmed culture medium before transferring them into the reactor using the Pasteur pipette. Magnetically stabilized continuous flow bioreactor (MSCFB)

The reactor was made of a hollow glass tube, and both of its ends were closed with rubber septa which stabilized an inner glass rod located longitudinally in the middle of the reactor. Two or three neodymium magnets separated by PDMS spacer(s) were fixed half way along the outside of the glass tube, and cells attached on collagen coated MABs were introduced to the center of the reactor to stabilize them in the magnetic field. Fresh medium flows from the reservoir to the reactor through the pump, and the waste medium flows from the reactor back to the reservoir (FIG. 1B).

One end of the inner glass rod was fitted into the septum and the rod was inserted into the outer reactor glass tube using the free end of the rod, and sealed the opening. Then, the free end of the rod was fitted with septum and kept without sealing the opening. The magnet was mounted on the outside of the reactor tube at half way of the length of the tube. Pre-warmed culture medium was added into the reactor to about 1 cm above the magnet using the Pasteur pipette, and then the cell-attached magnetic beads were added into the reactor, and allowed to settle and form the circular shape above the magnet. A PDMS spacer was placed on top the magnet and the second magnet was mounted onto the first one before adding more beads into the reactor. If the reactor was full, the culture medium above the beads was removed to create space and add more beads for the third magnet. Once all the beads were inserted, the reactor was completely sealed with septum and transferred it into the incubator. The reservoir flask was filled with 130 ml of pre-warmed culture medium and the flow system was set up to start the flow.

Unsterile components to be used in the bioreactor system were sterilized by autoclaving. The system was set up in the cell culture hood, and the openings of the tubing were closed with capped needles while autoclaving the system and the caps were removed just before the needles were inserted into the reactor.

The medium to be used in the bioreactor system was warmed in 37° C. water bath before using it in the bioreactor. If the pump was located outside the incubator, it is especially important to warm the medium to avoid condensation on the silicone tubing inside the incubator. After sterilizing, the bioreactor was clamped vertically in the cell culture hood and opened the top septum to add the pre-warmed culture medium to approximately halfway or just above the magnet. Then, the magnetic beads with cells seeded on them were slowly added into the reactor using the 1 ml pipette, and let them evenly spread out to form the first ring of magnetic beads on top of the magnetic field just above the magnet. The ring of magnetic beads was moved downward into middle of the magnetic field by slightly lifting the magnet up and put the magnet back in the original position before the second ring of beads was formed just above the magnet. The top septum was closed again after filling the culture medium into the bioreactor, and transferred it into the incubator. The sterilized reservoir was filled with culture medium and was also transferred into the incubator and ran the pump to remove the air column in the tubing before connecting the reservoir to the bioreactor. Once all the connections were made, the continuous flow of culture medium was started in the closed loop. The connections were wrapped with Parafilm to reduce the air exposure, and hence reduce the chance of contamination. One such configuration of the bioreactor is described below.

### Components and Dimensions of an Exemplified MSCFB

| Components | Description | Dimension | Quantity |
|---|---|---|---|
| Reactor tube | Hollow | L: 160 mm, OD: 10 mm, | 1 |
| | | ID: 8 mm | |
| Inner rod | Solid | L: 155 mm, Dia: 3 mm | 1 |
| Magnet | Neodymium | H: 0.25 in, OD: 1 in | 3 |
| | | ID: 0.5 in | |
| PDMS Spacer | Donut shape | H: 0.25 in, OD: 1 in | 2 |
| | | ID: 0.5 in | |
| Septum for reactor | White | OD: 8 mm | 2 |
| Hypodermic needle | Purple | 16G 1.5 in | 4 |
| Silicone tubing | Fresh culture | L: 12.5 ft, OD: 0.25 in, | 1 |
| (Reserv.-React.) | medium | ID = 0.125 in | |
| Silicone tubing | Waste | L: 3.5 ft, OD: 0.25 in, | 1 |
| (React.-Reserv.) | medium | ID = 0.125 in | |
| Septum for reservoir | White | 24/40 joint | 1 |
| Filter for ventilation | Acrodisc | 0.2 μm pore, 25 mm | 1 |
| Erlenmeyer Flask | Pyrex | 125 ml | 1 |
| reservoir | | | |
| Syringe needle for | Metal hub | 9 in | 1 |
| reservoir outlet | | | |

### Histological Processing of Tissue Tube Produced in MSCFB

The tissues taken of the bioreactor were fixed in 10% neutral buffer formalin for 6 hours and embedded into paraffin. Five micron tissue sections were cut and stained in hematoxylin and eosin (H&E; reagents from Richard Allan Scientific, Kalamazoo, Mich.).

### Magnetic Agarose Beads (MABs)

Magnetic agarose beads were prepared by the emulsification method. The size and shape of the MABs were controlled by adjusting the physical and chemical parameters such as the stirring speed and concentration of agarose during the process. They could be separated by size using the deposition method in the presence of an external magnetic field. The main ingredients of the MABs are inert to the cellular environment and thus MABs could be used to attach cells after derivatizing the surface, without affecting the stability of the beads. The surface of the MABs could be chemically derivatized for immobilization of biomolecules such as collagen or poly-L-lysine to facilitate seeding with desired type of cells.

### Surface Modification of MABs

Collagen fibers were immobilized on the carboxylated surfaces of MABs via EDC-NHS coupling. To achieve a coating of collagen on the surface, collagen solution was diluted in deionized water. The attachment was verified by using fluorescent collagen. It was found that the collagen coating on the surface could be achieved without aggregating collagen if the coating process was performed in the acidic solution, and thus cell attachment could also be achieved on the collagen coated MABs without forming cells clumps due to aggregated collagen.

MABs were coated with fluorescent collagen after derivatizing the MABs' surfaces with COOH groups. In the cells, the collagen layer could be observed as green fluorescence, confirming that a thin coating of collagen on the surface was deposited by this immobilization technique. Extracellular matrix (ECM) of RASM tissue contains collagen, and in order to allow the cells to produce their own ECM, only a thin layer of collagen coating is desired on the surface

### Cell Immobilization of Rat Aortic Smooth Muscle Cells (RASMCs) on MABs

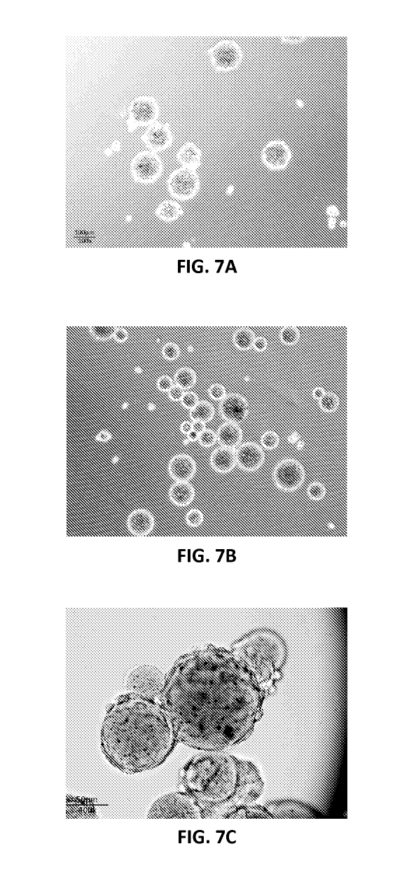

Attachment of the smooth muscle cells, an adherent cell line, on the round beads was contemplated. One method for achieving this goal was agitation by rotation for 9 hours at 37° C. The MABs coated with collagen coating seeded with RASMC are shown in FIG. 7A, microscopic image showed that the cells adhered to the beads and had proliferated on them. In this image, the cells can be seen as small bright hemispheres on the surface of the spherical MABs. Approximately 10-50 cells were attached on an average size bead of 100 μm in diameter. A cluster of cells may sometimes attach to two beads linking the beads together.

Agitation by rotation at 10 rpm ensured that most of the cells would not attach to the walls of the test tube, and that the chances of contact between cells and beads were increased. After 9 hours of incubation most of the beads had cells attached on them without forming aggregates of beads. Although some cells might cluster together during the incubation process, they were removed afterwards before transferring the cell-immobilized beads into the bioreactor for the tissue culture in the magnetic field. FIG. 7B shows that modified MABs without collagen coating kept in the same experimental conditions had almost no cells attached on the beads after the same time period.

Higher magnification images showed that the RASM cells were immobilized on the collagen coated MABs and that the surfaces of most of the beads were well covered with cells after this period of incubation (FIG. 7C). At a shorter incubation time, it was found that both the number of cells attached onto MABs and the number of MABs with attached cells was smaller. At a longer incubation time, the attached cells on the MABs overgrew and served as links to the other beads forming a cluster of beads, which hindered the initial seeding of the bioreactor to form a circular shape of beads, and also it was hard to separate the cluster without damaging the seeded cells on the surface.

It was also observed that the RASM cells could adhere onto the MABs and proliferated well on spherical surface of the MABs without adhering to the surface of the test tube. On the other hand, the cells tend to anchor the MABs down onto the culture tube or dish surface in the case of stationary cell seeding method without rotation. Therefore, the advantages of rotation method were that cells could freely proliferate on the entire surface of the beads and that the suspending MABs with immobilized cells could be easily transferred into the MSCFB to allow for tissue growth.

### Magnetically Stabilized Continuous Flow Bioreactor (MSCFB)

The MSCFB was assembled as described above shows the set up of the MSCFB built in the cell culture incubator. Culture medium in the system starts from the reservoir which consists of a 125 ml Erlenmeyer flak, and is pumped back into it after entering into the bioreactor from the and exiting from the top as shown in the CAD on the right (FIG. 1B). Hypodermic needles were used at the tubing connections to create a closed loop system. The neodymium ring magnets stabilizing the MABs with attached cells were placed about halfway between the inlet and outlet of the bioreactor. A 0.2 μm syringe filter attached to the needle inserted in the septum of the growth medium reservoir served as the vent for gas exchange between the culture medium and surrounding air.

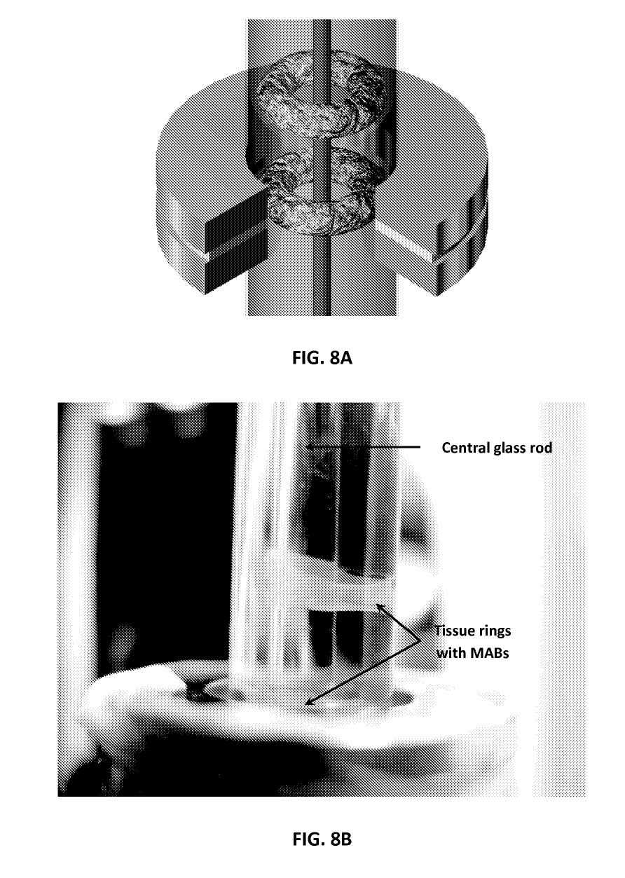

The presence of the ring magnets resulted in the formation of two distinct rings of magnetic beads inside the glass tube of the bioreactor, as shown in the diagram in FIG. 8A. The same assembly of the magnetic agarose beads was observed even when they were loaded with cells. It was expected that upon cell and tissue growth these rings of cells would later be linked to form a tubular shape tissue facilitated by the upward flow of culture medium in the system.

The central glass rod acted as an accessory to our bioreactor system in generating the tubular shape of the tissue, as RASM cells normally tend to contract once they started forming tissue and they would end up forming an irregular-shape tissue mass in the absence of the central glass rod. The MABs were important in serving as support to initiate the formation of tubular-shape tissue on the central rod.

After 3-4 day incubation tissue rings were observed forming around the central glass rod inside the MSBFB just where the magnetic bead rings had formed initially (FIG. 8B). These tissue rings were formed from cells seeded on MABs. MABs were observed on the entire tissue ring.

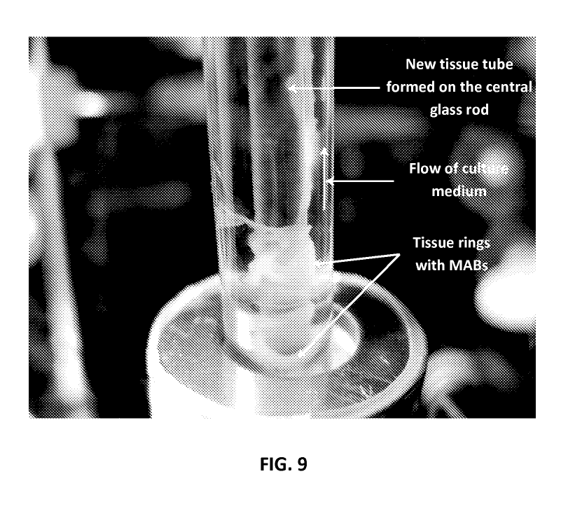

When these two rings of tissues were formed, there was no tissue formation above and below these tissue rings or between the two rings of tissues. The new tissue rings were first formed on the inside wall of the bioreactor away from the central glass rod as observed on the right side of the tissue ring shown in FIG. 9. Once the tissue ring contracted as mentioned earlier, it detached from the surface of the bioreactor and settled onto the central glass rod without any obvious shift in vertical position. At this point, the entire tissue ring was supported by the magnetic field and no other piece of tissue was observed possibly because these new tissue rings formed by linking the cells attached to MABs had not yet fully adhered onto the central glass rod or the cells had not proliferated on the central glass rod.

After one-week incubation, the tissue rings fully contracted onto the central rod and tissue growth was observed along tube forming upward in the direction of the flow of culture medium. As shown in FIG. 9, both tissue rings can be seen and they're both relatively thicker than the newly formed tissue tube on the surface of the central rod. It is important to note that the tissue tube formed outward from the tissue ring contained no magnetic bead. At this point, irregular small outgrowths of tissue were also observed since some of the tissue pieces without the MABs detached from the main tissue rings. When a piece of tissue detached from the main structure, it could usually take any of the three possible routes. First, if the entire piece of tissue completely detached from the main structure, it would usually sink toward the bottom of the bioreactor since the detached tissue piece usually contained no MAB and thus was not stabilized by the magnetic field, and the force of the flow culture medium exerted onto the tissue piece was not strong enough to hold the entire falling piece of tissue against the gravity. If the tissue piece was still partially attached to the main tissue structure on one side or if the free end of the tissue piece was light enough to be lifted up by the flow of culture medium, the free end of the piece would usually land onto the central rod above the main tissue structure, which served as a pivot. On the other hand, if the flow of culture medium could not carry the free end of the tissue piece, it would hang downward and land onto the central glass rod.

Removing the Tissue Tube from the MSCFB

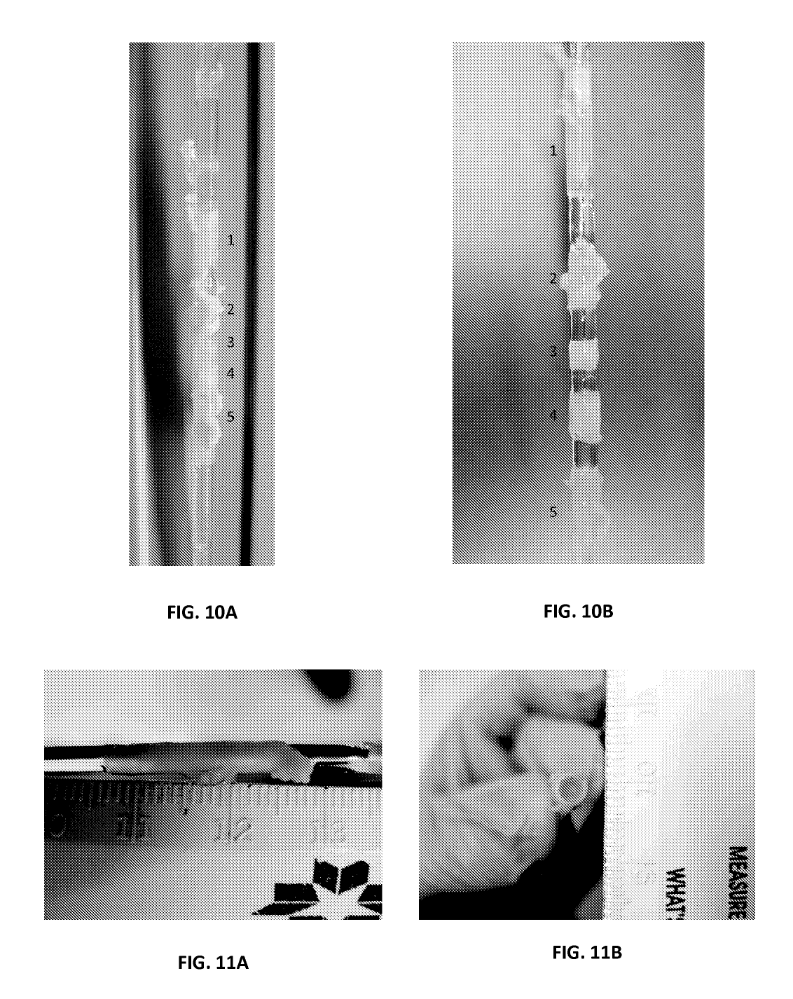

FIGS. 10A and 10B show the 19-day old RASM tissue tube taken out of the MSCFB. Tissue rings were first formed on the inside wall of the bioreactor from the cells attached on MABs the magnetic beads on which the cells initially proliferated, and the tissue tube was formed above and between the two tissue rings, since the initial tissue rings were subjected to flow of culture medium from the bottom to the top. The observed tissue tube below the tissue ring possibly grew from a hanging tissue piece shed from the main structure and landed onto the central glass rod. The entire tissue tube is about 70 mm long.

To be able to characterize the generated tissue tube by histological methods, i was cut into 5 separate pieces: #1, #3, and #5 were the sections of the tissue tube without the MABs, and #2 and #4 were the sections containing the MABs. In these figures, extra tissue pieces on the tissue tube could be observed. They were formed by detaching from the original tissue rings and reattaching onto the central glass rod due to the gravity and the flow of culture medium.

FIG. 11A shows the RASM tissue tube section without the MABs. This section of tissue tube was about 24 mm long and its inner diameter reflected the diameter of the central glass rod, which had 3 mm diameter. Before the tissue was processed, it was fixed in 10% neutral buffer formalin for at least 2 hours and stored in PBS overnight at 4° C. After fixation, the tissue could safely be handled without damaging its integrity and it was later transferred onto an agarose rod to start the embedding process.

FIG. 11B shows a section of the RASM tissue tube transferred onto an agarose rod before embedding in paraffin and slicing it. The agarose rod had the same diameter, 3 mm, as the central glass rod, and was made by 4% agarose in diH₂O using the 3 mm inner diameter gas tubing as a mold. Each piece of agarose rod was cut into appropriate length to hold different pieces of tissue to be processed. Inserting agarose rod inside the tissue tube helped maintain the size and shape of the tissue and prevent it from shrinking during the repeated dehydration and rehydration process. The agarose rod used for this purpose was flexible and did not interfere with the sectioning nor had negative effect on the microtome.

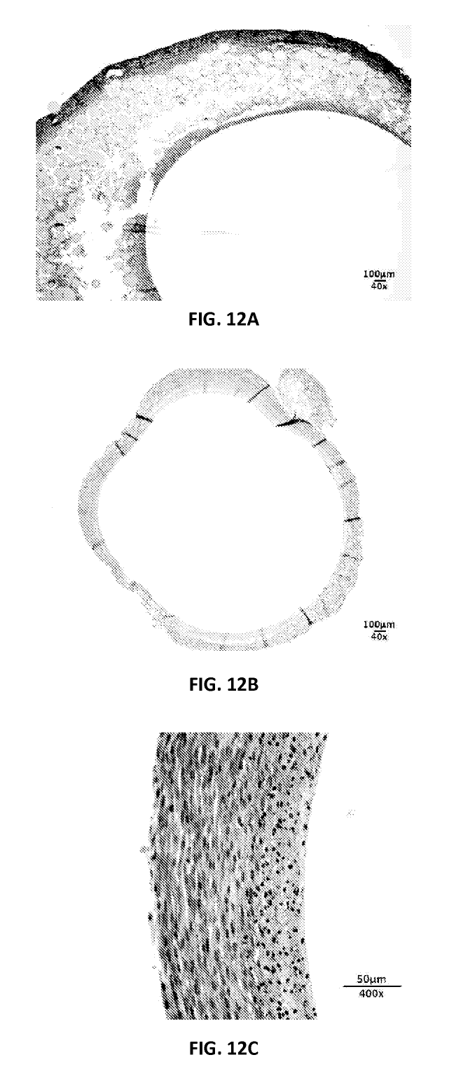

FIG. 12A shows the H&E (Hematoxylin and Eosin) stain of 29-day old tissue ring with MABs grown inside the magnetic field of the MSCFB. In color, the purple color shows the nuclei of the cells and the pink color shows the cytoplasm of the cells, and grey color shows the MABs. It was found that the healthy cells were located on the exterior edge of the tissue, and the cells among the beads and on the interior edge exhibited sign of necrosis. The scattered white spaces inside the tissue may occur during the staining process because the beads were not as held strongly each other due to necrosis in that region.

FIG. 12B shows a section of the 29-day old RASM tissue tube without the MABs. This was the portion of the tissue tube grew from the tissue ring with MABs. Since the tissue without the MABs was not as thick as that with the MABs, the gas and nutrient diffuse better and thus the minimum necrosis occurred in this portion. The dark transverse lines across the tissue were the creases of tissue slice appeared during the mounting and staining process.

FIG. 12C shows the close-up of the RASM tissue ring without the MABs grew from the tissue ring with MABs. The majority of the cells in the tissue tube without the MABs were relatively healthier than those with the MABs, and the tissue was about 30 cell-layers thick. The tissue tube as positioned vertically when it was embedded into paraffin so that the ring structure could be visible when the tissue was sectioned to stain. High magnification shows that cells in the tissue ring were circumferentially aligned perpendicular to the direction of the flow although the tissue tube was extended upward in the direction of the flow of culture medium, and thus they could radially contract towards the lumen of the tissue tube.

### Example 5

### Interleukin-2 (IL-2) Production in the MSCFB of Example 4 with Three Magnetic Rings

Jurkat cells were seeded without agitation on collagen coated agarose magnetic beads in 37° C. with 5% CO₂ using the T-25 tissue culture flask for 3-6 days. Jurkat cells are non-adherent type but after a few days most of the beads attached with cells. At the end of the incubation, unattached cells were aspirated by collecting the beads with attached cells on the bottom of the flask using a magnet. They were resuspended by pre-warmed culture medium and transferred into the sterilized bioreactor setup (as described above) with 3 magnets separated by magnetic repulsion to create 3 rings of cells in the magnetic field. After 3 days of incubation, the control sample was taken out of the reactor and 50 ng/ml of PMA was added to trigger the production of IL-2 from the Jurkat cells. After 48 hours of incubation in PMA, the experimental sample was taken. The samples were lyophilized and re-concentrated in smaller volume to be able to detect by human IL-2 ELISA detection kit.

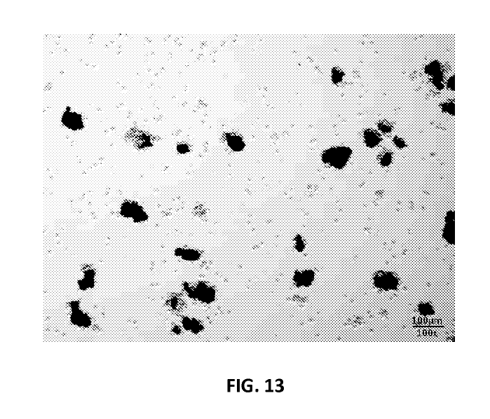

Jurkat cells attached on the collagen coated rough MABs that had an irregular shape or tough surface. see FIG. 13. This demonstrates the potential applications of the system by varying the types of beads used. These types of beads were found to immobilize a larger number of suspended cells, such as Jurkat cells, compared to smooth round beads. The cells were cultured in the presence of modified MABs for up to about 1 week to increase the number of cells attached to beads before transferring into the bioreactor.

Unlike RASM cells, which must adhere to a surface for survival and proliferation, Jurkat cells normally remain in suspension during the entire culture period. Rough MABs also helped Jurkat cells stay attached on the beads longer than those on the smooth surface of the spherical MABs. Due to their suspension nature, Jurkat cells tend to come off from the MABs as they proliferate, and thus they were incubated in the presence of the beads for about 3-6 days prior to transferring into the MSCFB.

### Results

Three ring magnets were assembled in parallel so that they could trap a relatively large number of beads without interfereing with each others magnetic fields and thus maximize the capacity of the bioreactor. The jurkat cells were seeded on collagen coated MABs in tissue culture flasks for 3-7 days for to allow for maximum and enhanced attachments of cells before transferring into the MSCFB. Since the Jurkat cells were not contractile cells, the central glass rod was not necessary in the set up. The rest of the components were assembled as in the tissue culture MSCFB of Example 4, and the flow of culture medium was also from the bottom of the bioreactor to the top.

### Interleukin-2 (IL-2) Production in the MSCFB

Since Jurkat cells were shown to be immobilized on rough agarose beads, Jurkat cells were seeded in the bioreactor and their ability to produce IL-2 while trapped in the magnetic was assayed as described above. IL-2 is a native protein of the immune system which has successfully been used as a drug for certain types of cancer. Jurkat cells produce little to no IL-2 under normal conditions, but when stimulated by PMA the production of IL-2 is activated. The ELISA results showed that 48 hours after introduction of PMA in the bioreactor, the IL-2 level in the lyophilized sample was 20±5 pg/mL, whereas in the sample taken prior to PMA it was 5±1 pg/mL Although the amount of IL-2 produced in this particular experiment is relatively low due to small cell concentration compared to the total growth medium volume in the bioreactor, this result indicates that our incubator can potentially be used for the continuous production of IL-2 or other cell metabolites.

### Summary

Growing 3D tissue in the magnetically-stabilized, continuous-flow bioreactor (MSCFB) was shown in the Examples. Rat aortic smooth muscle cells (RASMC) were successfully seeded onto magnetic agarose beads (MABs) through biochemical modification of the agarose surface. The RASMC tissue rings with MABs were initially grown in suspension inside the magnetic field, out of which the RASM tissue tube without the MABs grew mainly due the influence of the flow of culture medium. Based on histological staining of RASM tissue product, it was found that the tissue tube grew out of the initial tissue rings with MABs contained healthy and highly proliferating cells. Suspension cells such as Jurkat cells were demonstrated to seed onto rough MABs and were stabilized by the magnetic field inside the continuous flow bioreactor.

These results demonstrate the potential of using a bioreactor such as configured above for the generation of tissue in a desired shape and also continuous production of cell metabolites. It is noted that the principles of bioreactors can be developed by one of skill in the art to produce other desired tissues in various forms. The MCCFB allows for external control of the tissue shape by modifying and manipulating the magnetic field. A bioreactor system will also easily allow for the addition of other cell types or layers of the same or different cell types resulting in the generation of more complex tissues and potentially organs. The continuous production of cell products including (cells themselves) removes the need for harvesting and re-seeding of the bioreactors. This can allow for a controlled growth of the tissue or a controlled process to generate cell products. Such technology and advances in this field of research additionally have applications in the production of biofuels from microorganisms.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A bioreactor for culturing cells, comprising:
a vessel,
an apparatus for conveying fluids,
magnetic beads having cells attached thereon, and
an apparatus for conveying a magnetic field.

2. The bioreactor of claim 1, further comprising
an apparatus for dialysis, and
an apparatus for harvesting.

3. The bioreactor of claim 1 further comprising an apparatus for photoelectrochemical processing.

4. (canceled)

5. (canceled)

6. The bioreactor of claim 1, where the magnetic bead size is 100 nm to 10 μm.

7. The bioreactor of claim 1, wherein more than one type of cell is attached.

8. (canceled)

9. (canceled)

9a. (canceled)

9b. (canceled)

10. A system for cultivating cells, comprising:
a bioreactor,
magnetic beads having cells attached thereon,
an apparatus for conveying a magnetic field, and
an apparatus for conveying a continuous flow of fluids, wherein the cells are controlled by the magnetic field and the flow of fluids.

11. (canceled)

12. (canceled)

13. A method for culturing cells in a bioreactor comprising:
providing cells attached to magnetic beads,
acting upon the cells with a fluid,
acting upon the cells with a magnetic field, and
allowing the cells to grow and proliferate.

14. The method of claim 13, wherein the cells form a tissue.

15. The method of claim 14, wherein the tissue has a lumen.

16. The method of claim 13, wherein the cells are grown without scaffolding.

17. The method of claim 13, wherein the cells are mammalian cells.

18. The method of claim 17, wherein the mammalian cells are selected from lymphocytes, smooth muscle cells, stem cells, cardiac cells and fibroblasts.

19. The method of claim 13, wherein the magnetic beads are coated with a polymer.

20. The method of claim 19, wherein the polymer is agarose.

21. (canceled)

22. The method of claim 20, where in the magnetic bead is modified by attachment of a protein.

23. The method of claim 22, wherein the protein is collagen or antibody.

24. The method of claim 13, wherein the cells grow to form tissue rings.

25. The method of claim 13, wherein the magnetic field is produced from angular magnetic rings and the magnetic beads.

26. The method of claim 13, wherein the magnetic field is varied.

27. (canceled)

28. (canceled)

29. (canceled)

30. (canceled)

31. A method for making a three dimensional cell tissue comprising,
culturing cells attached to magnetic beads in a bioreactor,
acting upon the cells with a fluid,
acting upon the cells attached to magnetic beads with a magnetic field, and
allowing the cells to grow and proliferate into a three dimensional cell tissue.

32. The bioreactor of claim 1, wherein the magnetic field is varied.
